(19) United States
(12) Patent Application Publication
(10) Pub. No.: US 2025/0009699 A1
(43) Pub. Date: Jan. 9, 2025

(54) LIPID EMULSIONS FOR PARENTERAL NUTRITION

(71) Applicants: BAXTER INTERNATIONAL INC., Deerfield, IL (US); BAXTER HEALTHCARE SA, Glattpark (Opfikon) (CH)

(72) Inventors: Abdelatif Elouahabi, Brussels (BE); Zouaoui Bourezg, Braine I'Alleud (BE); Baptiste Bodet, Braine I'Alleud (BE); Nathalie Cospin, Brussels (BE); Jean-Luc Trouilly, Braine I'Alleud (BE); Mary Hise Brown, Vernon Hills, IL (US); Thibault Senterre, Chaudfontaine (BE)

(21) Appl. No.: 18/217,821

(22) Filed: Jul. 3, 2023

(51) Int. Cl. A61K 31/202; A61J 1/10; A61K 9/00; A61K 9/107; A61K 31/355; A61K 31/575; A61K 31/683; A61K 47/44; A61P 1/16; A61P 3/02

(52) U.S. Cl. CPC A61K 31/202; A61J 1/10; A61K 9/0029; A61K 9/107; A61K 31/355; A61K 31/575; A61K 31/683; A61K 47/44; A61P 1/16; A61P 3/02

(57) ABSTRACT

The present disclosure relates to improved lipid emulsions for providing parenteral nutrition, including ready-to-use parenteral nutrition formulations comprising such lipid emulsions. More particularly, the present disclosure is directed to improved lipid formulations or emulsions including multi-chamber containers comprising same, wherein the lipid emulsion contains DHA, EPA, and ARA in an optimized concentration and ratio, optionally in combination with choline and defined levels of phytosterols. The present disclosure further relates to methods of avoiding and/or treating liver damage and/or inflammation, and to methods for improving fatty acid profiles in plasma and certain tissues or organs especially of pediatric patients.

### TECHNICAL FIELD

The present disclosure relates to improved lipid emulsions for providing parenteral nutrition, including ready-to-use parenteral nutrition formulations comprising such lipid emulsions. More particularly, the present disclosure is directed to improved lipid formulations or emulsions including multi-chamber containers comprising same, wherein the lipid emulsion contains DHA, EPA, and ARA in an optimized ratio optionally in combination with choline and defined levels of phytosterols. The present disclosure further relates to methods of avoiding and/or treating liver damage and/or inflammation especially in pediatric patients receiving parenteral nutrition, and to methods for improving fatty acid profiles in plasma and certain tissues and/or organs of the pediatric patients, including retina, liver, and lung.

### DESCRIPTION OF THE RELATED ART

Lipids are a key energy source for patients who require parenteral nutrition, including newborn infants and provide for the essential omega-3 and omega-6 fatty acids that the human body is unable to produce. Lipids also provide important long-chain essential polyunsaturated fatty acids (LC-PUFAs) that are crucial for normal development of the central nervous system and other organ systems (Burrin et al., 2014, Impact of New-Generation Lipid Emulsions on Cellular Mechanisms of Parenteral Nutrition-Associated Liver Disease. Advances in Nutrition 5, 82-91). n-3 and n-6 PUFAs have received specific attention for their role in brain development and cellular functions. Docosahexaenoic acid (DHA, 22:6n-3) and arachidonic acid (ARA, 20:4n-6), for example, are always present in human milk. These fatty acids are discussed as key players in the structure and function of human tissues, immune function, and brain and retinal development especially during gestation and infancy. There is ongoing research into the potential of, for example, omega-3 PUFA and the benefit of enriching enteral nutrition (EN) and parenteral nutrition (PN) formulations with these compounds, with varying outcomes (see, for example, Singer et al., 2021, Enteral and supplemental parenteral nutrition enriched with omega-3 polyunsaturated fatty acids in intensive care patients-A randomized, controlled, double-blind clinical trial. Clinical Nutrition 40, 2544) that underline the persistent challenge of identifying and providing optimized PN formulations despite being aware of some or most of the key components in the nutrition of, for example, pediatric patients in need.

There are various lipid emulsions on the market, including soybean oil-based or soybean oil/safflower oil-based formulations (Intralipid, Liposyn II), wherein Intralipid, for example, is enriched with certain fatty acids. Both contain phytosterols which are natural components of vegetable oils. More recent lipid emulsions for parenteral nutrition are based on olive oil and soybean oil (e.g., ClinOleic), fish oil (e.g., Omegaven) or blends of soybean oil, olive oil, MCTs and fish oil (e.g., SMOFlipid). Lipid emulsions are either provided as such or are part of multichamber nutritional products that further comprise, in separate chambers, carbohydrate and/or amino acid formulations that are admixed with the lipid emulsion formulation upon administration. The DHA, EPA, and ARA content of these available lipid emulsions varies, depending, for example, on the source of the oils used for preparing the lipid emulsions. ARA and DHA have been added to infant formulas in the United States since 2001. In Europe, supplementation began at an even earlier time. Most infant formulas contain 0.2% to 0.4% of total fatty acids as DHA and between 0.35% and 0.7% of total fatty acids as ARA based on worldwide averages of DHA and ARA content in human milk. Human milk has a mean (±SD) concentration of DHA in breast milk (by weight) of 0.32±0.22% and a mean (±SD) concentration of ARA of 0.47±0.13%, indicating that the ARA concentration is on average lower than that of DHA (Brenna et al., 2007, Docosahexaenoic and arachidonic acid concentrations in human breast milk worldwide. The American Journal of Clinical Nutrition. 85 (6), 1457).

It is known that DHA (22:6n-3) and ARA (20:4n-6) are present in human milk and play important roles in the structure and function of human tissues and immune function. Preterm infants who are born at a stage when the normal placental transfer and deposition of DHA in fetal tissues, for example, is not yet completed, DHA seems to be especially relevant.

Also, the role of EPA and ARA have been discussed in the literature regarding metabolic health and risk of developing certain PN-related diseases such as inflammation.

Generally, the need for fulfilling the nutritional needs of a patient must be balanced against certain risks associated with a prolonged or extensive parenteral nutrition, including, for example, the development of cholestasis as a key element of PN-associated liver disease (PNALD) in infants. Cholestatic liver disease is connected with certain increased serum biochemical markers, such as bilirubin, g-glutamyl transpeptidase, bile acids, and liver transaminases, and in some patients, steatosis may occur (Burrin et al., 2014, Advances in Nutrition 5, 82-91). However, it remains unclear how exactly the parenteral nutrition with lipid emulsions and their respective composition contributes to said liver disease and other health issues that are generally connected with receiving parenteral nutrition. It is, therefore, difficult to develop optimized lipid emulsions and currently there are no proven effective therapeutic approaches.

Identifying optimized lipid emulsions in terms of oil sources and combinations, fatty acid profiles, or the presence or absence, and the reduction or addition of compounds such as, for example, certain vitamins, phytosterols, choline, and other compounds that have been discussed in the literature remains an important goal and challenge that the present disclosure addresses.

There are many publications that address the above question and that contemplate the benefit of various lipid emulsion compositions and the presence especially of compounds like DHA, EPA, ARA, but also of phytosterols, choline and/or α-tocopherol. For example, the above-mentioned publication of Burrin et al., 2014, Advances in Nutrition 5, 82-91, discusses the above compounds and their influence on liver disease.

Molina et al., 2020, New generation lipid emulsions increase brain DHA and improve body composition, but not short-term neurodevelopment in parenterally-fed preterm piglets. Brain, Behavior, and Immunity 85, 46-56, discuss key fatty acids such as docosahexaenoic acid (DHA), eicosapentaenoic acid (EPA), and arachidonic acid (ARA), their content in parenteral lipid emulsions and their influence on brain development, inflammation, and behavior of piglets. Specifically, the publication provides for a comparison of enterally fed piglet versus piglets receiving parenteral nutrition with Intralipid, SMOFlipid and an experimental composition comprising DHA (10.82 mg/mL), ARA (6.59 mg/mL), and EPA (1.97 mg/mL).

Hadley et al., 2016, The Essentiality of Arachidonic Acid in Infant Development. Nutrients 8, 216, review the role of arachidonic acid (ARA) for infant development. It is stated in the publication that there is strong evidence based on animal and human studies that ARA is critical for infant growth, brain development, and health, but that it is also important to correctly balance the amounts of ARA and DHA as too much DHA may suppress the benefits provided by ARA.

WO2020007758A1 discloses lipid emulsions for parenteral administration comprising soybean oil, medium-chain triglycerides, olive oil, and fish oil, wherein the lipid phase comprises less than 25 mg campesterol, less than 30 mg stigmasterol and less than 120 mg beta-sitosterol per 100 g of the lipid phase, and wherein the fish oil comprises at least 35 wt.-% DHA and preferably less than 10 wt.-% EPA based on the total weight of the fish oil. The lipid emulsions may further comprise 1-10 wt.-% arachidonic acid (ARA).

WO2019232044A1 broadly discloses parenteral nutrition formulations for pediatric patients including lipid emulsions comprising DHA and EPA in a ratio of 10:1 to 1000:1 (w/w) or which are completely free of EPA, and wherein phytosterols are present in an amount of less than 70 mg per 100 g of the oil phase. Also disclosed are lipid emulsions comprising DHA and ARA in a ratio of from 10:1 to 1:5 (w/w) as well as formulations comprising DHA, ARA, and EPA. The formulations may also contain choline.

### SUMMARY

In light of the disclosure herein, and without limiting the scope of the invention in any way, in a first aspect of the present disclosure, which may be combined with any other aspect listed herein unless specified otherwise, a lipid formulation for parenteral administration to a patient is disclosed, which comprises an aqueous phase and from about 5% to 35% by weight of an oil phase based on the total weight of the lipid emulsion (w/w), and which is characterized by a specific combination of DHA, EPA, and ARA, that has shown to be especially beneficial with regard to maintaining or improving liver health and reducing liver damage, respectively, reduced inflammation and/or improved deposition of ARA and/or DHA in certain important tissues such as the brain, lung, liver and/or retina.

In the context of the present disclosure, it was found that specific ratios between DHA, EPA, and ARA, all of which are compounds generally known for playing important roles in the nutrition of patients, especially also of pediatric patients who receive lipid emulsions as part of their parenteral nutrition scheme. Specifically, it was found that lipid emulsions preferably comprising from about 2.0 g/L to about 15.0 g/L of DHA, from about 0 g/L to about 1.2 g/L of EPA, and from about 5.0 g/L to about 20.0 g/L of ARA, show clear benefits over other composition of the same type but different ratios or concentrations.

It could be shown that the compositions disclosed herein can improve certain relevant markers that describe potential liver injury compared to other lipid emulsion which also contain DHA, EPA, and/or ARA, but which are different in their specific composition and ratios between these compounds. At the same time, the lipid emulsions disclosed herein show a clear improvement of the fatty acid profile in plasma and certain tissues, such as, for example, retina, liver, and lung, over other lipid emulsions.

According to one aspect of the lipid emulsions disclosed herein, the ratio between DHA and ARA preferably is from 1:0.5 to 1:2. EPA can be present in the lipid emulsions but should remain at a low level compared to DHA and ARA as defined above. According to one aspect, the ratio of DHA to EPA preferably is from 30:1 to 10:1.

According to certain aspects of the invention, EPA can be absent from the lipid emulsions disclosed herein, which means that EPA concentrations are below the limit of detection (LOD) when analyzed with known methods for determining EPA.

According to another aspect, the lipid emulsions of the invention also comprise choline in an amount of from 0.5 g/L to 5.0 g/L. Choline has the potential to beneficially influence the above-stated effect on the liver, inflammation and/or fatty acid profile of certain tissues and/or the deposition of ARA and/or DHA in certain tissues. Preferably, choline is selected from the group of choline derivatives consisting of choline chloride and glycerophosphocholine (GPC) but can be added to the lipid emulsion of the invention also in other forms.

According to yet another aspect of the invention, the lipid emulsion preferably comprises phytosterols in an amount not exceeding 200 mg/L of the lipid emulsion. According to another aspect, phytosterols will be present in a concentration not exceeding 150 mg/L, preferably not exceeding 140 mg/L, and especially preferably not exceeding 130 mg/L.

According to a further aspect of the invention, the lipid emulsions disclosed herein may further contain linoleic acid in a concentration of from 25 to 50 g/L, and/or α-tocopherol in a concentration of from 100-300 mg/L.

According to another aspect of the invention, the ratio of ω-6:0-3 fatty acids preferably is from 4:1 to 2:1.

The lipid emulsion according to the present disclosure are especially suitable for pediatric patients, where liver damage and inflammation during parenteral nutrition remain an issue, and where the best possible development of tissue and organs such as lung, liver, retina, and brain are of the essence. However, also adolescents and adult patients will benefit from the lipid emulsions disclosed herein.

According to another aspect, the invention relates to a method of treating patients, especially pediatric patients, that suffer from or are at risk of suffering from liver damage and/or inflammation due to parenteral nutrition. According to yet another aspect, the invention relates to a method of providing parenteral nutrition to pediatric patients to support the adequate development of the central nervous system and tissues such as retina, liver, lung, and brain and/or to pediatric patients who are at risk of suffering from dietary fatty acid imbalances.

According to one aspect of the present invention, the lipid emulsions disclosed herein can be standalone products for the parenteral nutrition of a patient in need. In this case, the lipid emulsions can be provided in a single chamber bag or container that is designed for such individual administration of the lipid formulation to a patient. Single-chamber containers can be, for example, flexible, rigid, or semi-rigid containers as well as glass vials.

According to one aspect of the invention, the lipid emulsion according to the invention is directly administered to the patient without admixing the lipid emulsion with other parenteral nutrition formulations, such as, for example, amino acid formulations or carbohydrate formulations.

According to another aspect of the invention, the lipid emulsion can be provided as a component of a multipart parenteral nutrition product, such as a multi-chamber container that in addition to the lipid emulsion provides for a carbohydrate or an amino acid formulation or both in adjacent chambers of the container. The lipid emulsion as disclosed herein will be admixed with said other formulations before administration by breaking the seals between the respective chambers. According to another aspect of the invention, a multi-chamber bag comprising a lipid emulsion according to the invention can further comprise electrolytes, vitamins or trace elements or combinations thereof either as part of any of the lipid, carbohydrate, or amino acid formulation or as formulations provided in one or more separate chambers. For example, the MCB may have 2, 3, 4, 5, 6 or more chambers. Generally, the MCB will have two, three, or four chambers. The chambers of said MCB may have the same size or may have different sizes to accommodate various compositions and volumes. The chambers may be designed to contain volumes of from, for example, 1 to 5 ml, from 5 to 10 ml, from 10 to 50 ml, from 50 to 100 ml, from 100 to 250 ml, from 250 ml to 500 ml, from 500 to 1000 ml, or from 1000 to 1500 ml. The MCBs can be designed to have chambers which are located adjacent to each other. The chambers may have various shapes. The chambers can be oriented horizontally and/or vertically to each other. Certain small chambers can be designed to be located within another, larger chamber, wherein, for example, the small chamber, which is located within another, larger chamber can be accommodated and fixed into said larger chamber by welding at least one edge of said small chamber in between the weld seam of the surrounding larger chamber.

According to another aspect, the multi-chamber container includes frangible barriers between the chambers which can be opened before use to mix the components of all or selected chambers.

According to another aspect, the lipid emulsion provided in a multi-chamber bag comprises DHA, EPA, and ARA in a concentration that will result in concentrations of from about 2.0 g/L to about 15.0 g/L of DHA; from about 0 g/L to about 1.2 g/L of EPA; and from about 5.0 g/L to about 20.0 g/L of ARA in the final admixture with other components in the MCB. The respective amounts can be easily calculated based on the intended final volume of the admixture and can be adjusted, for example, via the concentration in the lipid emulsion and/or the volume of the lipid emulsion which is part of the MCB.

According to yet another aspect of the present invention, DHA, EPA, and ARA are provided in a solution which has been reconstituted from a multi-chamber bag and wherein DHA, EPA, and ARA are present in a concentration of from about 2.0 g/L to about 15.0 g/L of DHA; from about 0 g/L to about 1.2 g/L of EPA; and from about 5.0 g/L to about 20.0 g/L of ARA.

According to one embodiment, the lipid emulsion as disclosed herein is provided in a dual chamber bag or a three-chamber bag comprising at least one of an amino acid formulation and a carbohydrate formulation.

According to another aspect of the invention, the lipid emulsion or reconstituted solution according to the invention is administered by means of a central or peripheral catheter.

According to another aspect of the present invention, a method for providing parenteral nutrition to a patient, preferably a pediatric patient, is provided, wherein the patient requires parenteral nutrition and wherein the parenteral nutrition supports the adequate development of the central nervous system, especially of the brain, as well as the adequate development of critical tissues such as retina, liver, and lung, and wherein the patients is administered a lipid emulsion according to the present invention either alone or in combination with other nutritional formulations.

According to another aspect of the invention, the patient receiving the lipid emulsion disclosed herein requires parenteral nutrition because oral and enteral nutrition is not possible, insufficient, or contraindicated. According to one aspect, the patient needs receiving parenteral nutrition which supports an adequate development of the central nervous system, especially the brain, as well as an adequate development of critical tissues such as retina, liver, and lung, and/or is at risk of suffering from dietary fatty acid imbalances.

According to a further aspect, the patients preferably treated with the lipid emulsions disclosed herein suffer from or are at risk of developing liver disease and inflammation.

It was found that the lipid emulsions as defined herein have the potential to reduce inflammation and liver damage that is often associated with parenteral nutrition, especially in pediatric patients. This finding is surprising given that especially ω-6 fatty acids like ARA that are contained in higher levels in certain vegetable oils like soybean oil are currently discussed as having pro-inflammatory effects leading to liver damage and increased inflammation (Clader et al., 2020, Lipids in Parenteral Nutrition: Biological Aspect. JPEN 44, Supplement 1, S1-S27), whereas higher EPA levels are suggested to be beneficial due to the anti-inflammatory effects of this fatty acid.

However, it could be shown here that, surprisingly, higher ARA levels in combination with higher DHA levels and reduced levels of EPA, potentially in the presence of choline and well balanced phytosterol levels seem to result in an unexpected reduction of markers that are otherwise associated with liver injury and may further have the potential to also reduce inflammation. It was further found in the course of the present work, that a difference should be made between phytosterols as defined herein, i.e., phytosterols that are typically contained in vegetable oils, and desmosterol, which is a typical component of algae oils.

Specifically, it was found in the course of the work leading to the present invention, that certain levels of phytosterols in combination with desmosterol do not seem to have a negative impact on liver injury and inflammation. Rather, if the phytosterol level (as defined herein) remains below a certain threshold, higher levels of desmosterol have no negative impact on inflammation and liver damage. Accordingly, the impact of phytosterols as defined herein and desmosterol, both together referred to as “total phytosterol” should be carefully scrutinized with regard to any perceived impact on liver injury and/or inflammation.

Finally, it could be shown herein that while, surprisingly, inflammation and liver damage could be kept at bay or could even be reduced with the proposed lipid emulsions, the higher ARA levels contained in said lipid emulsions could be translated into a higher ARA deposition in tissues that are specifically relevant for parenterally fed pediatric patients, such as lung and retina, compared to state of the art compositions.

### BRIEF DESCRIPTION OF THE DRAWINGS

Understanding that figures depict only embodiments of the invention and should not be considered limiting to the scope of the present disclosure, the present disclosure is described and explained with additional specificity and detail by using the accompanying figure.

FIG. 1 provides for the plasma levels of linoleic acid in plasma fatty acid analyses according to Example 4. The Figure compares the results found in the animal model described in Example 2 for the group parenterally fed with a lipid emulsion according to the present invention (“NGF 2”)) and those fed with known lipid emulsions (“Intralipid” and SMOFlipid”). The Control group consists of sow-reared piglets. The level of linoleic acid is higher for the “NGF 2”-group than in the other groups, including the Control group.

FIG. 2 provides for the plasma levels of alpha-linolenic acid in plasma fatty acid analyses according to Example 4. The Figure compares the results found in the animal model described in Example 2 for the group parenterally fed with a lipid emulsion according to the present invention (“NGF 2”)) and those fed with known lipid emulsions (“Intralipid” and SMOFlipid”). The Control group consists of sow-reared piglets. The level of linoleic acid is comparable to the other parenterally fed groups, whereas are all are lower than the Control group.

FIG. 3 provides for the plasma levels of arachidonic acid in plasma fatty acid analyses according to Example 4. The Figure compares the results found in the animal model described in Example 2 for the group parenterally fed with a lipid emulsion according to the present invention (“NGF 2”)) and those fed with known lipid emulsions (“Intralipid” and SMOFlipid”). The Control group consists of sow-reared piglets. The level of arachidonic acid is higher compared to the “Intralipid” and “SMOFlipid” group, and even exceeds the values found for the Control group.

FIG. 4 provides for the plasma levels of eicosapentaenoic acid in plasma fatty acid analyses according to Example 4. The Figure compares the results found in the animal model described in Example 2 for the group parenterally fed with a lipid emulsion according to the present invention (“NGF 2”)) and those fed with known lipid emulsions (“Intralipid” and “SMOFlipid”). The Control group consists of sow-reared piglets. The level of eicosapentaenoic acid is lower than the one found in “SMOFlipid” and the Control. The Intralipid group has even lower EPA levels.

FIG. 5 provides for the plasma levels of docosapentaenoic acid (DHA) in plasma fatty acid analyses according to Example 4. The Figure compares the results found in the animal model described in Example 2 for the group parenterally fed with a lipid emulsion according to the present invention (“NGF 2”)) and those fed with known lipid emulsions (“Intralipid” and SMOFlipid”). The Control group consists of sow-reared piglets. The level of DHA is about the same level as in the Control group, higher in the “Intralipid” group, and somewhat lower than in the “SMOFlipid” group.

FIG. 6 shows the results for all groups of the animal study regarding bile flow (mcg/g liver) as a measure for liver function. Groups “Intralipid” and “SMOFlipid” have been parenterally fed with the respective commercial lipid formulations, see Example 5 and Example 5.1. The “NGF 2” group refers to the group having been parenterally fed with a lipid formulation according to the invention (“NGF 2”). Bile flow is highest in the Control group (sow-reared piglets), whereas bile flow in the “NGF 2” group is almost as good as in the Control group and better than in the “Intralipid” and “SMOFlipid” group.

FIG. 7 shows the results for all groups of the animal study regarding “total bilirubin” (mM/L) as a measure for liver function. Groups “Intralipid” and “SMOFlipid” have been parenterally fed with the respective commercial lipid formulations, see Example 5 and Example 5.2. The “NGF 2” group refers to the group having been parenterally fed with a lipid formulation according to the invention (“NGF 2”). Higher bilirubin levels indicate liver injury. Bilirubin levels are very low in the “NGF 2” group and basically the same as the found in the Control group. The comparative test groups “Intralipid” and “SMOFlipid” are both higher, with a more pronounced increase in bilirubin in the “Intralipid” group.

FIG. 8 shows the results for all groups of the animal study regarding “unconjugated bilirubin” (mM/L) as a measure for liver function. Groups “Intralipid” and “SMOFlipid” have been parenterally fed with the respective commercial lipid formulations, see Example 5 and Example 5.2. The “NGF 2” group refers to the group having been parenterally fed with a lipid formulation according to the invention (“NGF 2”). An increase in conjugated bilirubin is typically associated with inflammation and liver injury. The NGF 2 group shows very low levels of conjugated bilirubin which is at about the same level as the Control group. The comparative lipid emulsions “Intralipid” and “SMOFlipid” result in clearly higher levels of conjugated bilirubin in the tested groups.

FIG. 9 shows the results for all groups of the animal study regarding “bile acids” as a measure for liver function. Groups “Intralipid” and “SMOFlipid” have been parenterally fed with the respective commercial lipid formulations, see Example 5 and Example 5.3. The “NGF 2” group refers to the group having been parenterally fed with a lipid formulation according to the invention (“NGF 2”). Liver injury (cholestasis) is associated with higher bile acid levels. Bile acid levels are very low in study group “NGF 2”. The comparative groups “Intralipid” and “SMOFlipid” as well as the Control group have higher bile acid levels.

FIG. 10 shows the influence of the lipid formulations on the gamma-glutamyltransferase (GGT) activity levels in the respective tested groups, see Example 5.5. Comparative test groups “Intralipid” and “SMOFlipid” have been parenterally fed with the respective commercial lipid formulations, “NGF 2” refers to the group having been parenterally fed with a lipid formulation according to the invention (“NGF 2”), and “Control” refers to the sow-reared group. High levels of GGT activity can be a sign of bile duct damage. GGT activity is lowest in the Control group similarly low in the study groups, with stably low values for the “NGF 2” group in comparison with the “SMOFlipid” group.

FIG. 11 shows the influence of the lipid formulations on the aminotransferase (ALT) activity levels in the respective tested groups, see Example 5.4. Comparative test groups “Intralipid” and “SMOFlipid” have been parenterally fed with the respective commercial lipid formulations, “NGF 2” refers to the group that was parenterally fed with a lipid formulation according to the invention (“NGF 2”), and “Control” refers to the sow-reared group. Increased levels of ALT activity can be a sign of liver damage. ALT activity is similar in the study groups over the study period with the exception of the Control group where it is found to be highest.

FIG. 12 shows the influence of the lipid formulations on the transaminase (AST) activity levels in the respective tested groups, see Example 5.4. Comparative test groups “Intralipid” and “SMOFlipid” have been parenterally fed with the respective commercial lipid formulations, “NGF 2” refers to the group that was parenterally fed with a lipid formulation according to the invention (“NGF 2”), and “Control” refers to the sow-reared group. AST levels are higher for all parenterally fed study groups compared to the Control, but are lowest for NGF 2, indicating its positive impact on the liver.

FIG. 13 shows the results for the various study groups in terms of their histology score. The severity of liver damage as deduced from tissue samples can be graded as further explained in Example 6. A higher HistoScore indicates more degenerative findings. FIG. 13 shows that parenteral nutrition with a lipid emulsion according to the invention (“NGF 2” group) scores lowest of all parenterally fed groups and is closest to the Control group with normally raised piglets.

### DETAILED DESCRIPTION

Certain embodiments described herein relate generally to the field of parenteral nutrition. More particularly, some embodiments described herein relate to lipid emulsions for parenteral administration, wherein the lipid emulsion comprises an aqueous phase and an oil phase and is present in the form of an oil-in-water emulsion. Lipid emulsions as discussed herein are provided as stand-alone lipid emulsions for parenteral administration but can also be provided together with other nutritional formulations, such as amino acid formulations or carbohydrate formulations for parenteral nutrition. For example, the lipid emulsions disclosed herein can be contained in multi-chamber containers for parenteral administration, wherein the containers comprise said lipid emulsion in a first chamber and a carbohydrate formulation or amino acid formulation in a second chamber, or optionally an amino acid in a third chamber when the second chamber contains a carbohydrate formulation. The respective chambers may further comprise vitamins and/or electrolytes and/or trace elements. Said vitamins and/or amino acids can also be provided in separate chambers of said multi-chamber bags, such as, for example, in a third and/or fourth and/or fifth chamber, respectively.

The term “pediatric” refers to neonates, including premature (pre-term), full term, and post-mature neonates of up to one month of age; the term “infants” specifically refers to persons of between one month and one year of age. If not expressly indicated otherwise, the term “pediatric” as generally used herein encompasses infants. The term “children” refers to persons between one and up to 12 years of age. The term “adolescents” as used herein refers to persons of between 13 and up to 21 years of age. The term “adult” as used herein refers to persons of 22 years of age and older.

As used herein, the term “essentially free” may refer to a composition that contains no more than 5% of the specified component, no more than 3%, no more than 2%, no more than 1%, no more than 0.58, no more than 0.18, no more than 0.05%, no more than 0.02%, no more than 0.01%, no more than 0.005%, no more than 0.002%, and/or no more than 0.001% of the specified component. For example, a lipid emulsion or composition that is “essentially free of EPA” may refer to a composition that contains no more than no more than 2%, no more than 1%, no more than 0.5%, no more than 0.1%, no more than 0.05%, no more than 0.02%, no more than 0.01%, no more than 0.005%, no more than 0.002%, or no more than 0.001% EPA. The term “no EPA” means that the content of EPA is below the level of detection.

The expressions “liver disease”, “liver injury” and “liver damage” are interchangeably used herein. The expression refers to a condition of the liver which is defined by one or more of reduction of bile flow, increase of total bilirubin, unconjugated bilirubin, and/or conjugated bilirubin, increase in bile acids, increased activity of alkaline phosphatase (AP), increased activity/levels of gamma-glutamyl transferase (GGT), increased activity/levels of aspartate transaminase (AST), increased activity/levels of alanine aminotransferase (ALT), reduced bile flow, and histologically determined signs of liver injury.

The expression “LC-PUFA” refers to long-chain polyunsaturated fatty acids having 220 carbon atoms and 23 double bonds, including the biologically active LC-PUFAs, such as EPA, DHA, and ARA. The expression “PUFA” generally refers to polyunsaturated fatty acids having two or more double bonds. LC-PUFAs are believed to play a central role in cellular structure and function, including the regulation of membrane fluidity, cell signaling, and protein expression, promotion, and suppression of tissue immune and inflammatory responses as well as organogenesis. The most well-known functions of LC-PUFAs in the developing fetus comprise supporting brain and retina development (Lapillonne and Moltu, 2016, Long-Chain Polyunsaturated Fatty Acids and Clinical Outcomes of Preterm Infants. Ann Nutr Metab 69:36-44).

The expression “ω-3 fatty acid(s)”, also called omega-3 oils, omega-3 fatty acids or n-3 fatty acids, refers to polyunsaturated fatty acids (PUFAs) characterized by the presence of a double bond, three atoms away from the terminal methyl group in their chemical structure. The omega-3 fatty acids involved in human physiology are α-linolenic acid (ALA), eicosapentaenoic acid (EPA) and docosahexaenoic acid (DHA).

The expression “ω-6 fatty acid(s)”, also called omega-6 fatty acids, omega-6 oils or n-6 fatty acids, refers to polyunsaturated fatty acids with a final carbon-carbon double bond in the n-6 position, that is, the sixth bond, counting from the methyl end. Arachidonic acid is an example of an omega-6 (n-6) polyunsaturated fatty acid. Its precursor is linoleic acid (LA). Vegetable oil is a major dietary source of omega-6 fatty acids.

The expression “phytosterol(s)” as used herein, if not expressly indicated otherwise, refers to vegetable oil based phytosterols (plant sterols). The expression specifically refers to stigmasterol, sitosterol, avenasterol, and campesterol. Sterols such as, for example, desmosterol, are not covered by this definition. Where it is intended to cover phytosterols and other sterols such as desmosterol, the expression “total phytosterols” is used. Procedures for the qualitative and quantitative analysis of sterols, including phytosterols, in lipid emulsions such as used in parenteral nutrition are known in the art. They generally consist of a preliminary extraction of the total lipids with an organic solvent, followed by saponification and isolation of the unsaponifiable fraction, which is then analyzed using a variety of procedures including enzymatic, colorimetric, capillary gas liquid chromatographic (GC), and high-performance liquid chromatographic (HPLC) techniques. For example, phytosterols as described herein can be determined by GC and HPLC, which are reliable, selective, and accurate methods to assay the sterol content in parenteral lipid emulsions. The methods are described, for example, in Xu et al., 2012, Steroidal Compounds in Commercial Parenteral Lipid Emulsions. Nutrients 4:904-921.

The expression “about” as used herein in connection with the definition of concentration or ranges means that small deviations from the values disclosed are encompassed. As used herein, “about” or “approximately” and the like, when used in connection with a numerical variable, can generally refers to the value of the variable and to all values of the variable that are within the experimental error (e.g., within the 95% confidence interval for the mean) or within +/−10%, preferably within +/−5% and especially preferably within +/−1% of the indicated value. As used herein, the term “about” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, reflecting tolerances, conversion factors, rounding off, measurement error and the like, and other factors known to those of skill in the art such that equivalent results or effects are obtained. In general, an amount, size, formulation, parameter or other quantity or characteristic is “about” whether or not expressly stated to be such. It is understood that where “about” is used before a quantitative value, the parameter also includes the specific quantitative value itself, unless specifically stated otherwise.

The present disclosure provides lipid formulations for parenteral administration comprising DHA, ARA and EPA in a defined concentration and ratio which could be shown to have superior effects in comparison with other lipid emulsions comprising the same components. Advantages could be determined based on liver injury typical markers, such as, for example, bile flow, GGT activity, histopathology, inflammation, fatty acid deposition, as further detailed in the Example section. Specifically, ARA deposition was improved in relevant tissues such as lung, liver and retina, and DHA deposition could be shown to be improved at least in lung and retina.

Lipid emulsions having the improved effects as described above are characterized by comprising an aqueous phase and about 5% to about 35% by weight of an oil phase based on the total weight of the lipid emulsion (w/w) and comprising from about 2.0 g/L to about 15.0 g/L of DHA, from about 0 g/L to about 1.2 g/L of EPA, and from about 5.0 g/L to about 20.0 g/L of ARA. According to one embodiment of the invention, the ratio between DHA and ARA is from 2:1 to 1:2 (or 1:0.5 to 1:2).

The ratio between DHA and ARA can vary within a certain defined range as defined above. Certain embodiments of the invention are based on a ratio of DHA:ARA from 1:0.75 to 1:1.8, from 1:0.7 to 1:1.6, from 1:0.8 to 1:1.5, from 1:0.8 to 1:1.4, from 1:0.8 to 1:1.2 or from 1:0.9 to 1:1.1. A ratio of DHA:ARA of about 1:1 is another embodiment of the lipid emulsion according to the invention. As will be readily understood, small deviations of from 2% to 5% from this ratio are encompassed by the present disclosure.

At the same time, the absolute concentrations of DHA and ARA as well as EPA can independently from each other vary within the ranges given above.

According to certain embodiments, the DHA concentration according to the invention can be from about 3.0 g/L to about 14.0 g/L, from about 4.0 g/L to about 13.0 g/L, from about 4.0 g/L to about 14.0 g/L, from about 5.0 g/L to about 14.0 g/L, from about 6.0 g/L to about 13.0 g/L, from about 7.0 g/L to about 13.0 g/L, from about 8.0 g/L to about 12.0 g/L, or from about 9.0 g/L to about 12.0 g/L.

The ARA concentration in the lipid emulsion according to the invention will be selected, relative to the DHA concentration, according to the ratios of DHA and ARA as defined before.

According to certain embodiments, the ARA concentration in the lipid emulsion according to the present invention can be from about 5.0 g/L to about 19.0 g/L, from about 5.0 g/L to about 18.0 g/L, from about 6.0 g/L to about 17.0 g/L, from about 6.0 g/L to about 16.0 g/L, from about 6.0 g/L to about 15.0 g/L, from about 7.0 g/L to about 14.0 g/L, from about 8.0 g/L to about 13.0 g/L, from about 8.0 g/L to about 12.0 g/L, or from about 9.0 g/L to about 12.0 g/L.

The EPA concentration in the lipid emulsion according to the invention will be within the range as defined before. According to certain embodiments, the EPA concentration according to the invention is from about 0 g/L to about 1.2 g/L of EPA, from about 0.1 g/L to about 1.2 g/L of EPA, from about 0.2 g/L to about 1.0 g/L of EPA, from about 0.3 g/L to about 1.0 g/L of EPA, from about 0.4 g/L to about 1.0 g/L of EPA, from about 0.4 g/L to about 1.0 g/L of EPA, from about 0.5 g/L to about 1.0 g/L of EPA, or from about 0.6 g/L to about 1.0 g/L of EPA. According to another specific embodiment, the lipid emulsion according to the present invention does not contain any EPA, which means that EPA is contained below the level of detection.

Methods for determining the presence or levels of EPA in a lipid emulsion are known in the art. For example, EPA as well as other fatty acids, such as, for example, DHA and ARA, can be determined using GC-MS chromatography, HPLC and LC-MS. For example, GC-MS can be used for determining EPA, such as described in Driscoll et al., 2009, Pharmacopeial compliance of fish oil-containing parenteral lipid emulsion mixtures: Globule size distribution (GSD) and fatty acid analyses. International Journal of Pharmaceutics 379:125-130. Preferably, the method is in accordance with “Lipid injectable emulsion.” In: United States Pharmacopeia and National Formulary. United States Pharmacopeial Convention; 2008.

In some cases, DHA, ARA, and EPA (if any) is present in triglyceride form or in ethyl ester form, preferably in triglyceride form.

According to certain embodiments, the lipid emulsions disclosed herein may also contain choline. It is known that choline plays a central role in the development of pediatric patients as it is an essential structural component of cell membranes, the neurotransmitter acetylcholine and phospholipid synthesis, where it contributes to the synthesis of very low-density lipoprotein necessary for formal triglyceride exportation from the liver. Choline is connected, especially in infants, with brain development. Choline deficiency is thought to activate cellular apoptosis, which may contribute to a deficient cell repair mechanism. Choline deficiency is discussed as a reason for hepatic steatosis, which is especially of concern in infants, including pre-term babies, who require parenteral nutrition. In the context of the present invention, choline has been found to have the potential to enhance the positive effects found for the present lipid emulsions comprising certain combinations and amounts of DHA and ARA, and potentially increase the deposition of e.g., ARA in the relevant tissues such as, for example, lung, liver and/or retina, while generally improving liver health especially of the pediatric patients treated with a lipid emulsion according to the present invention.

Choline can be added to the lipid emulsion according to the present invention in different forms, such as, for example, choline chloride, CDP-choline (sodium salt), glycerophosphocholine (GPC), choline bitartrate, to the extent it can be stably added to a lipid emulsion since some forms of choline are preferably soluble in aqueous solutions. According to one embodiment of the invention, the choline derivative glycerophosphocholine (GPC), (2-{[(2S)-2,3-dihydroxypropyl phosphonato]oxy}ethyl)trimethylazanium, which is also referred to as glycerophosphorylcholine, aglycerophosphocholine, L-alpha-glycerylphosphorylcholine, choline alfoscerate or α-glycerophosphocholine, is a choline derivative which can advantageously be used in parenteral formulations of the present invention as an efficient choline source, such as described already in WO20190232054A1. According to one embodiment of the invention, GPC is added in an amount of from 0.5 g/L to 5.0 g/L. Other choline forms can be used in the same equivalent amounts.

According to one specific embodiment, choline can be added in an amount from 0.5 g/L to 4.5 g/L, from 0.5 g/L to 4.0 g/L, from 0.5 g/L to 3.5 g/L, from 0.5 g/L to 3.0 g/L, from 1.0 g/L to 5.0 g/L, from 1.0 g/L to 4.5 g/L, from 1.0 g/L to 4.0 g/L, from 1.0 g/L to 3.5 g/L, from 1.0 g/L to 3.0 g/L, from 1.5 g/L to 4.5 g/L, from 1.5 g/L to 4.0 g/L, from 1.5 g/L to 3.5 g/L, from 1.5 g/L to 3.0 g/L, from 2.0 g/L to 5.0 g/L, from 2.0 g/L to 4.5 g/L, from 2.0 g/L to 4.0 g/L, or from 2.0 g/L to 3.5 g/L.

Phytosterols also play a role in the lipid emulsions of the present invention. As mentioned before, the expression “phytosterol(s)” refers to vegetable or plant oil based phytosterols (plant sterols), and specifically refers to stigmasterol, sitosterol, avenasterol, and campesterol. Other sterols, such as desmosterol, a cholesterol precursor, are not encompassed by the expression. If phytosterols and other sterols, such as desmosterol, are addressed as a whole, the expression “total phytosterol(s)” is used. For example, soybean oil has a high content of important fatty acids such as linoleic acid (LA) and α-linoleic acid (ALA), whereas olive oil is a source especially for LA and oleic acid. Therefore, lipid emulsions based on soybean oil or mixtures of soybean and olive oil are commonly used in parenteral nutrition. A possible complication of long-term parenteral nutrition can sometimes be the occurrence of parenteral nutrition-associated liver disease (PNALD), especially affecting pediatric patients. There are indications that PNALD could be related to parenteral uptake of phytosterols (PS). The absorption of dietary phytosterols by the oral route is very limited but can be higher during parenteral administration while the ability of the human body to metabolize phytosterols is limited.

According to one embodiment of the invention, the lipid emulsion comprises phytosterols in an amount not to exceed 200 mg/L of the lipid emulsion. According to another embodiment, the lipid emulsion comprises phytosterols in an amount not to exceed 180 mg/L, 160 mg/L, or 140 mg/L of the lipid emulsion. According to yet another embodiment, the lipid emulsion comprises phytosterols in an amount not to exceed 120 mg/L of the lipid emulsion. In some embodiments, the lipid emulsion comprises phytosterols in an amount not to exceed 100 mg/L of the lipid emulsion.

According to certain embodiments of the invention, the lipid emulsion may also contain desmosterol in an amount of up to 150 mg/L, up to 140 mg/L, up to 130 mg/L, or up to 120 mg/L of the lipid emulsion. Certain algae oils, such as, for example, oils derived from microalgae, possess some specific sterols which are generally not found in plant oils and are not considered phytosterols according to the definition as used herein. Besides fucosterol, desmosterol is typically found in algae oil and sometimes even predominates in certain algae types, such as red algae (Lopes et al., 2013: Sterols in Algae and Health (Chapter 9) in “Bioactive Compounds from Marine Foods: Plant and Animal Sources”, First Edition, Edited by Blanca Hernandez-Ledesma and Miguel Herrero. John Wiley & Sons, Ltd.). No immediate negative impact of desmosterol as a component of lipid emulsions used in PN is known so far. Phytosterols and desmosterol may both be present in the lipid emulsion according to the invention. In such case, they will be present in a total amount (of “total phytosterol”) not to exceed 250 mg/l of the lipid emulsion, wherein the above defined upper limits of phytosterol and desmosterol, respectively, will not be exceeded. Preferably, the total phytosterol amount will not exceed 240 mg/L, 230 mg/L, 220 mg/L, 210 mg/L, or 200 mg/L.

As mentioned above, linoleic acid (LA) is a relevant ω-6 fatty acid which is optionally present in the lipid emulsion according to the invention. The concentration of LA should be balanced to avoid too high contents of LA which are thought to be associated with higher oxidative stress especially in critically ill patients. According to one embodiment, the lipid emulsion according to the invention also comprises linoleic acid in an amount of from 25 to 50 g/L of the lipid emulsion, such as, for example, from 25 to 45 g/L, from 25 to 40 g/L, from 25 to 35 g/L, from 30 to 50 g/L, from 35 to 50 g/L, from 40 to 50 g/L, from 30 to 45 g/L, or from 30 to 40 g/L.

According to a further embodiment of the invention, the ratio of ω-6:0-3 fatty acids in the lipid emulsion is from 4:1 to 2:1. As mentioned before, eicosapentaenoic acid (EPA) and docosahexaenoic acid (DHA) are examples for ω-3 fatty acids, whereas arachidonic acid (AA) is a polyunsaturated ω-6 fatty acid. However, oils used for the preparation of lipid emulsions for PN contain further 0-6 and ω-3 fatty acids. Further ω-3 fatty acids found in oils used for the preparation of lipid emulsions such as in the present invention are, for example, alpha-linolenic acid (ALA), hexadecatrienoic acid (HTA), stearidonic acid (SDA), eicosatrienoic acid (ETE), eicosatetraenoic acid (ETA), heneicosapentaenoic acid (HPA), docosapentaenoic acid (DPA), and others. According to one embodiment of the invention, the group of fatty acids referred to as “ω-3 fatty acids” consists of alpha-linolenic acid (ALA), eicosapentaenoic acid (EPA) and docosahexaenoic acid (DHA). According to yet another embodiment of the invention, the group of fatty acids referred to as “ω-3 fatty acids” consist of eicosapentaenoic acid (EPA) and docosahexaenoic acid (DHA). The major ω-6 fatty acid is arachidonic acid (ARA) and its precursor linoleic acid (LA). LA can be metabolized to other ω-6 PUFAs and form, for example, ARA. According to one embodiment of the invention, the group of fatty acids referred to as “ω-6 fatty acids” consists of ARA and LA to which the expression ω-6 fatty acid refers in the context of the invention. The amounts of LA and ARA, respectively, are within the ranges as described above.

In the context of the present invention, it was found that the ratio of ω-6:ω-3 is of relevance for the effect of the lipid emulsion on improving the fatty acid profile and deposition of ARA especially in the lung, liver, and retina, and the positive effects on reducing inflammation and liver damage. The beneficial effects of the provision of ω-3 PUFAs (EPA and DHA) have been described before, including the finding that ω-3 PUFAs might also attenuate the negative effects on liver health of phytosterols present in plant-based LEs used in patients on long-term PN. However, nothing has been described for the ratio of ω-6:0-3 fatty acids in lipid emulsions that are designed as provided herein and which are specifically composed of DHA, ARA, and optionally EPA, choline and other components as disclosed herein. It was found that the ratio of ω-6:0-3 should preferably be from 4:1 to 2:1. According to another embodiment, the ratio is from 3:1 to 2:1 or from 4:1 to 3:1. In yet another embodiment the ratio is from 2:1 to 1:1. The amounts of DHA, EPA and ARA in any such ratio between ω-6 and ω-3 fatty acids is in each case within the ranges provided herein.

According to another embodiment of the invention, the lipid emulsion of the present invention comprises α-tocopherol in a concentration of from 100-300 mg/L of the lipid emulsion. α-tocopherol is the main form of vitamin E and generally used in lipid emulsions to restrict the peroxidation of susceptible PUFAs. α-tocopherol is also discussed for its ability to prevent or reduce liver damage, even though it does not seem to be effective alone against intestinal failure associated liver disease in infants, for example (Raphael and Duggan, 2012, Prevention and Treatment of Intestinal Failure-Associated Liver Disease in Children. Semin Liver Dis 32 (4): 341-347). The relevance and impact of the amount of α-tocopherol and its specific combination with other components in a lipid emulsion therefore remains open. It was found herein that α-tocopherol seems to have an additional positive impact when provided in a lipid emulsion as disclosed herein. According to some embodiments of the invention, the lipid emulsion comprises α-tocopherol in a concentration of from 120-280 mg/L, from 140-260 mg/L, from 160-240 mg/L, or from 180-220 mg/L of the lipid emulsion. According to a specific embodiment, α-tocopherol is added in an amount of about 200 mg/L of the lipid emulsion. These ranges in combination with the lipid emulsions other important components described above seem to be especially beneficial for improving the fatty acid, even though some currently available lipid emulsions already have α-tocopherol levels in that range. For example, the lipid emulsion ClinOleic (Baxter) has a α-tocopherol content of about 32 mg/L, SMOFlipid (Fresenius Kabi) has a α-tocopherol content of about 200 mg/L, Omegaven (Fresenius Kabi) has a α-tocopherol content of about 150-296 mg/L, and Intralipid (Fresenius Kabi) has a α-tocopherol content of about 38 mg/L (Raphael and Duggan, 2012).

In the present invention, lipid emulsions, multi-chamber containers comprising same, and compositions reconstituted from such multi-chamber containers are disclosed which are characterized by the presence of from about 2.0 g/L to about 15.0 g/L of DHA, from about 0 g/L to about 1.2 g/L of EPA, and from about 5.0 g/L to about 20.0 g/L of ARA in the lipid emulsion or the reconstituted composition as described above. The lipid emulsion will otherwise contain choline, phytosterols, linoleic acid and/or α-tocopherol as provided herein. The composition of said lipid emulsions can otherwise vary over a broad range as concerns specific components and/or the sources thereof, provided they can safely be used for intravenous administration to a patient in need of parenteral nutrition.

In certain embodiments, the lipid emulsions according to the invention are composed of an aqueous phase and about 5% to about 35% by weight of an oil phase based on the total weight of the lipid emulsion (w/w), wherein the lipid emulsion comprises from about 2.0 g/L to about 15.0 g/L of DHA; from about 0 g/L to about 1.2 g/L of EPA; from about 5.0 g/L to about 20.0 g/L of ARA; from about 120-280 mg/L α-tocopherol; from about 25 to 50 mg/L of linolenic acid; from 0.5 g/L to 4.5 g/L choline, preferably as choline chloride or GPC; and phytosterols in an amount of from 75 mg/L to 130 mg/L.

According to yet another embodiment, the lipid emulsions according to the invention are composed of an aqueous phase and about 5% to about 35% by weight of an oil phase based on the total weight of the lipid emulsion (w/w), wherein the lipid emulsion comprises from about 8.0 g/L to about 14.0 g/L of DHA; from about 0.1 g/L to about 1.0 g/L of EPA; from about 8.0 g/L to about 14.0 g/L of ARA; from about 150-250 mg/L α-tocopherol; from about 30 to 45 mg/L of linolenic acid; from 2.0 g/L to 5.0 g/L choline, preferably as choline chloride or GPC; and phytosterols in an amount of from 80 mg/L to 120 mg/L.

The lipid emulsions as disclosed herein are used in the treatment of patients who require parenteral nutrition. Patients who can benefit from the lipid emulsions according to the present invention can be adult patients and especially pediatric patients, wherein the DHA, ARA, and EPA concentrations as well as the concentrations and ratios of the other components can be adjusted as disclosed herein to address the needs of pediatric patients.

Pediatric patients that can especially benefit from lipid emulsions according to the invention are preterm babies and neonates who require parenteral nutrition and are at risk of developing inflammation and liver damage due to receiving parenteral nutrition. Accordingly, the lipid emulsions of the invention are suitable for treating or preventing liver damage especially in pediatric patients, including neonates, pre-term and extremely pre-term babies.

These patients also have specific needs in terms of their fatty acid profile which should allow for the adequate development of various tissues such as, for example, lung, retina, liver, intestines, and brain. The lipid emulsions according to the invention are suitable for avoiding or treating an imbalance in fatty acid profiles leading to an inadequate development of certain tissues such as, for example, lung and retina in neonates, including pre-term and extremely pre-term children. As mentioned before, fatty acids, especially including ω-6 and ω-3 PUFAs, play a central role in the development of said pediatric patients, as it is an essential structural component of cell membranes in tissues that may be underdeveloped or immature in the pediatric patients and especially in pre-term and extremely pre-term babies. The present invention therefore provides for a lipid emulsion for preventing and/or treating the inadequate development of relevant tissue due to an imbalance in the fatty acid profile of pediatric patients. Specifically, the lipid emulsion of the invention improves the deposition of DHA and ARA in said relevant tissues, including lung and retina.

However, the lipid emulsions of the invention or reconstituted compositions comprising same can also be used in the parenteral nutrition of adolescent or adult patients who are at risk of developing or suffer from liver damage, inflammation, and/or have specific needs as to an adequate fatty acid profile especially regarding ω-6 and ω-3 PUFAs and especially of DHA and ARA.

In certain embodiments the present lipid emulsions can also be used to provide the patients with an adequate amount of choline in order to address or prevent choline depletion or lack of choline as such or in order to provide choline as a component to synergistically improve the above-mentioned fatty acid profile. The presence of choline may also have a positive and synergistic effect on reducing or preventing the occurrence of inflammation and especially also on reducing or preventing liver damage.

As mentioned before, choline chloride and CDP-choline have already been used as choline derivatives for the supplementation of choline to patients. However, these compounds are not provided as a component of ready-to-use lipid emulsions for parenteral nutrition. If directly added to the lipid emulsion, the addition of choline chloride or CDP-choline to the formulation upon administration through the medical port or in a separate solution for intravenous application is not required, thereby avoiding additional steps which carry the risk of wrong dosage or contamination. In fact, it was found, that it is difficult to stabilize said choline derivatives in parenteral nutrition solutions, including lipid emulsions. According to certain embodiments of the invention, GPC is used for delivering choline to the patient in an effectively metabolizable form. GPC can further be stabilized in a lipid emulsion and remains stable in a formulation which is reconstituted from a multichamber bag comprising such lipid emulsion in one of the chambers. Accordingly, the invention also provides for a method of providing choline to a patient by parenteral nutrition as a component of a ready-to-use lipid emulsion or a reconstituted formulation comprising same, wherein the lipid emulsion comprises optimized amounts of DHA, ARA, EPA and optionally further components as disclosed herein.

Lipid formulations such as disclosed herein are an emulsion of an oil phase, a water phase, and an emulsifier that makes the two phases miscible. In case of lipid emulsions, which are to be used as an injectable emulsion for parenteral nutrition, the emulsion must be an oil-in-water (o/w) emulsion. This means that the oil must reside in the internal (or dispersed) phase, while water is the external (or continuous) phase, as the emulsion must be miscible with blood. Lipid emulsions as disclosed herein must therefore also be substantially free of any suspended solids. Of course, the lipid emulsions may contain components in addition to those disclosed above, including, but not limited to, antioxidants, pH modifiers, isotonic agents, vitamins, trace elements and various combinations thereof. An overview over lipid emulsions, their composition and use is provided, for example, in Driscoll 2017, Journal of Parenteral and Enteral Nutrition, 41, 125-134. Further information on the use of lipid emulsions in parenteral nutrition of intensive care patients is provided, for example, in Calder et al., 2010, Intensive Care Medicine, 36 (5), 735-749.

The oil phase of the lipid emulsion generally includes poly-unsaturated fatty acids in an ionized or salt form of the free acid, and/or in ester form. Suitable esters of the polyunsaturated fatty acids/long-chain polyunsaturated fatty acids include, but are not limited to, alkyl esters (e.g., methyl esters, ethyl esters, propyl esters, or combinations thereof) and triglyceride esters. In some cases, the long-chain polyunsaturated fatty acid has a structure R(C═O)OR′, wherein R is an alkenyl group having at least 17 carbon atoms, at least 19 carbon atoms, at least 21 carbon atoms, or at least 23 carbon atoms, and R′ is absent, H, a counter ion, an alkyl group (e.g., methyl, ethyl, or propyl), or a glyceryl group (e.g., R(C═O)OR′ is a monoglyceride, a diglyceride, or a triglyceride). Polyunsaturated fatty acids for use in the lipid formulations disclosed herein include, but are not limited to, linoleic acid (LA), arachidonic acid (ARA), α-linolenic acid (ALA), eicosapentaenoic acid (EPA), and docosahexaenoic acid (DHA) as disclosed above, which can accordingly be present in free acid form, ionized or salt form, alkyl ester form, and/or triglyceride form, and preferably in triglyceride form. In addition, stearidonic acid (SDA), γ-linolenic acid (GLA), dihomo-γ-linolenic acid (DPA), may be present in free acid form, ionized or salt form, alkyl ester form, and/or triglyceride form. In some cases, the majority or all of the polyunsaturated fatty acids and/or long-chain fatty acids are present in triglyceride form.

Typically, the lipid formulations disclosed herein include about 5% to about 35% by weight of an oil phase based on the total weight of the lipid emulsion. For example, the oil phase of the lipid emulsion is present in an amount of about 8% to 12%, of about 10% to about 20%, of about 10% to about 15%, of about 15% to about 20%, of about 12% to about 17%, of about 18% to 22% and/or about 20% by weight based on the total weight of the lipid formulation. The oil phase of a lipid emulsion typically and preferably contains, in various amounts depending on the source of the oil, omega-3 and omega-6 fatty acids. According to the present invention, at least the amounts of DHA, ARA, and EPA as well as the ratios between ω-6:0-3 fatty acids in the final lipid emulsion should be within the prescribed ranges and ratios to achieve the desired results.

The oil phase and its components can be derived from a single source or different sources (see, for example, Fell et al, Advances in Nutrition, 2015, 6 (5), 600-610). Of the plant oils, currently used sources include, but are not limited to, soybean and olive oil as well as coconut or palm kernel oil. Another source are algae, including microalgae such as Crypthecodinium cohnii and Schizochytrium sp., which in some cases serve as the single source of the long-chain polyunsaturated fatty acids docosahexaenoic acid (DHA) and/or ARA. Marine oil used in parenteral lipid emulsions is processed from oily fish primarily found in cold water and including, but not limited to, herring, shad, and sardines. However, other marine organisms can be used as an oil source, such as, for example, krill, such as Antarctic krill (Euphausia superba Dana). Krill oil, for example, provides for both EPA and DHA in amounts of up to 35% w/w of the fatty acids. Krill oil as a component of lipid emulsions is considered to have anti-inflammatory properties due to the presence of DHA and EPA and is hypothesized to bind endotoxins (Bonaterra et al., 2017, Krill oil-in-water emulsions protects against lipopolysaccharides-induced proinflammatory activation of macrophages in vitro. Marine Drugs, 15:74). According to the present invention, the amount of EPA should be strictly controlled and the ratio between DHA and EPA should be within the prescribed limits.

Many microbes including fungi, yeast and some bacteria can synthesize significant amounts of LC-PUFAs, mainly ARA (Sanaa et al., 2018, Journal of Advanced Research 11:3-13). Relevant ARA-producers are, for example, the nonpathogenic fungi Mortierella spp. from which the species M. alpina 15-4 and ATCC 32,222 produce ARA up to 70% of lipids. In some cases, the oil phase can include a blend of oils derived from the microalgae Schizochytrium sp. and the fungi Mortierella alpina. The oils in such a blend include, but are not limited to, myristic acid (C14:0), palmitic acid (C16:0), palmitoleic acid (C16:1), stearic acid (C18:0), oleic acid (C18:1), linoleic acid (C18:2 n-6), gamma-linolenic acid (C18:3, n-6), alpha-linolenic acid (C18:3, n-3), arachidic acid (C20:0), ARA (C20:4, n-6), EPA (C20:5, n-3), behenic acid (C22:0), DPA (C22:5, n-3), DHA (C22:6, n-3), and lignoceric acid (C24:0). In some cases, the blend includes ARA, DHA, and EPA. The content of various fatty acids such as DHA, EPA, ARA, linoleic acid (LA) or alpha-linoleic acid (ALA) in oils from different sources are summarized, for example, in Fell et al., 2015, Advances in Nutrition 6 (5): 600-610.

In some embodiments of the invention, the oil phase of the lipid emulsion according to the invention is essentially free of EPA but rich in ARA and DHA, wherein DHA and ARA are added alone or in combination to the oil phase of the lipid emulsion to reach the prescribed amounts and ratios, depending on the natural DHA and ARA levels of the oil used. EPA can also be present in the prescribed amounts either as a natural component of the oil used or as a component to be added in order to reach the desired final amount and ratios. Alternatively, the oil or oil blends used can be depleted of EPA.

The lipid emulsion of the invention includes certain amounts of ARA which may be present in the oil used for preparing the lipid emulsion and/or may be added to the formulation as needed to reach the desired final amounts and ratios. According to one embodiment, ARA used for preparing the lipid is obtained from a fungus, such as from Mortierella alpina. However, ARA can be obtained from various sources according to the present invention and is not limited to ARA derived from fungi. For example, ARA can be obtained as a component of oil blends.

In some embodiments of the invention, the oil phase also includes DHA and, optionally, EPA. If, according to an embodiment of the present invention, DHA is added to the formulation to reach the prescribed amounts, DHA can be obtained from algae, such as, for example, the non-photosynthetic marine micro-algae Crypthecodinium cohnii. However, DHA can be obtained from various sources according to the present invention.

Generally, oils for use in parenteral nutrition formulation such as disclosed herein and containing EPA, DHA, and/or ARA, may be processed and defined in compliance with and as further described in the GOED monograph requirements (GOED Voluntary Monograph, Volume 8.1, Issued Jan. 6, 2022.

An exemplary lipid emulsion according to the disclosure further includes choline in the form of glycerophosphocholine (GPC) in a concentration of from 0.1 g to 5.0 g per liter of lipid emulsion. According to another embodiment of the invention, GPC is present in the lipid emulsion in a concentration of from 0.5 g/L to 5.0 g/L.

In certain embodiments, the lipid emulsions disclosed herein in some cases have reduced levels of phytosterols. In such case, the oils used in the lipid emulsions may be depleted in phytosterols, typically by an amount of at least 25% of the total amount of phytosterols initially present in the oil, for example, at least 20%, at least 30%, at least 40%, at least 50%, at least 60%, or at least 75% depleted. In some embodiments, the lipid emulsions include 120 mg phytosterols or less per 100 g of oil phase, for example, about 50 mg to about 120 mg phytosterols per 100 g of oil phase, about 75 mg to about 100 mg phytosterols per 100 g of oil phase, and/or about 80 mg phytosterols to 110 mg per 100 g of oil phase. Removal or depletion of phytosterols can be carried out by known processes, for example, short path distillation, active charcoal treatment following by filtration, supercritical CO₂ chromatography, or chromatographic purification. For example, phytosterols can be removed as described in Ostlund et al., 2002, Am J Clin Nutr 75:1000-1004, or as described in U.S. Pat. No. 6,303,803 B1.

The lipid emulsions disclosed herein may further include additional components, such as surfactants (also referred to as emulsifiers), co-surfactants, isotonic agents, pH adjusters, and antioxidants. Generally, surfactants are added to stabilize emulsions by reducing the interfacial tension between the oil phase and the aqueous phase. Surfactants typically include a hydrophobic part and a hydrophilic part, and the amount of surfactant/emulsifier included in the formulations is determined based on the amount that is needed to achieve a desired level of stabilization of the emulsion. Typically, the amount of surfactant in the lipid formulation is about 0.01% to about 3% by weight based on the total weight of the lipid formulation, for example, about 0.01% to about 2.5%, about 0.01% to about 2.3%, about 0.02% to about 2.2%, about 0.02% to about 2.1%, about 0.02% to about 2%, about 0.05% to about 1.8%, about 0.1% to about 1.6%, about 0.5% to about 1.5%, about 0.8% to about 1.4%, about 0.9% to about 1.3%, about 1% to about 1.2%, and/or about 1.2% by weight. Suitable surfactants and co-surfactants include surfactants that are approved for parenteral use, and include, but are not limited to, phospholipids (e.g., egg phosphatide and soy lecithin), oleate salts, and combinations thereof. Krill oil can also be used as an emulsifier in the lipid emulsion, wherein the lipid emulsion comprises about 0.5 to 2.2 wt % krill oil based on the total weight of the emulsion, and wherein the emulsion is free of egg yolk lecithin (US 2018/0000732 A1). Another exemplary surfactant is lecithin, including both natural and synthetic lecithin, such as lecithins derived from egg, corn or soybean or mixtures thereof. In some cases, lecithin is included in an amount of about 1.2% based on the total weight of the lipid formulation.

In some cases, the lipid emulsion formulation includes a co-surfactant. Typically, the amount of co-surfactant in the lipid formulation is less than the amount of surfactant, and typically the amount of co-surfactant in the formulation is about 0.001% to about 0.6% by weight based on the total weight of the lipid formulation, for example, about 0.001% to about 0.55%, about 0.001% to about 0.525%, about 0.001% to about 0.5%, about 0.005% to about 0.5%, about 0.01% to about 0.4%, about 0.02% to about 0.3%, about 0.03% to about 0.2%, about 0.04% to about 0.1%, and/or about 0.05% to about 0.08%. An exemplary co-surfactant is oleate, such as sodium oleate. In some cases, the lipid formulation includes lecithin and oleate as surfactant and co-surfactant, for example, an in amount of 1.2% lecithin and 0.03% oleate. In some cases, sodium oleate is included in an amount of about 0.03% by weight based on the total weight of the lipid formulation.

Isotonic agents can be added to the lipid emulsions to adjust the osmolarity of the lipid emulsion to a desired level, such as a physiologically acceptable level. Suitable isotonic agents include, but are not limited to, glycerol. Typically, the lipid emulsion formulation has an osmolarity of about 180 to about 300 milliosmols/liter, such as about 190 to about 280 milliosmols/liter, and/or about 200 to about 250 milliosmols/liter. In some cases, the lipid emulsion includes an isotonic agent in an amount of about 1% to about 10% by weight based on the total weight of the lipid formulation, such as about 1% to about 5%, about 1% to about 4%, and/or about 2% to about 3%. In some cases, the lipid emulsion formulation includes about 2% to about 3% by weight of glycerol.

pH modifiers can be added to the lipid emulsions to adjust the pH to a desired level, such as a physiologically acceptable pH for parenteral use. Suitable pH modifiers include but are not limited to sodium hydroxide and hydrochloric acid. Typically, the lipid emulsion formulation has a pH of about 6 to about 9, such as about 6.1 to about 8.9, about 6.2 to about 8.8, about 6.3 to about 8.7, about 6.4 to about 8.6, about 6.5 to about 8.5, about 6.6 to about 8.4, about 6.7 to about 8.3, about 6.8 to about 8.2, about 6.9 to about 8.1, about 7 to about 8, about 7.1 to about 7.9, about 7.2 to about 7.8, about 7.3 to about 7.7, about 7.4 to about 7.6, about 7, about 7.5, and/or about 8.

The lipid formulations may further include antioxidants. Suitable antioxidants may be pharmaceutically acceptable antioxidants and include, but are not limited to, tocopherols (e.g., gamma tocopherol, delta tocopherol, alpha tocopherol), ascorbyl palmitate, or combinations thereof. In some cases, the lipid emulsion formulation includes an antioxidant in an amount of about 0 to about 200 mg/L, for example, about 10 to about 200 mg/L, about 40 to about 150 mg/L, about 50 to about 120 mg/L, about 75 to about 100 mg/L antioxidant(s), such as vitamin E. Where α-tocopherol is contained as an antioxidant in the lipid emulsion disclosed herein, it is part of the total amount of α-tocopherol present in the lipid emulsion as disclosed above, i.e., no additional α-tocopherol is provided to serve as an antioxidant.

The aqueous (or water) phase of all intravenous lipid emulsions must conform to the pharmacopeial requirements that make it suitable for injection, that is the water must be sterile water for injection.

As mentioned before, the lipid emulsions can be provided as such for parenteral administration. They can also be present as one of several formulations provided in a multi-chamber bag. The disclosure therefore also provides for a multi-chamber container for parenteral administration of nutritional formulations. For example, the container may be in the form of a bag having multiple compartments or chambers. The container, such as a bag, includes at least two chambers, but may also contain three, four, or five chambers, and in one preferred embodiment, two or three chambers. Suitable containers, including soft bags, typically are sterile, non-pyrogenic, single-use, and/or ready-to-use. The multi-chamber containers are particularly useful for holding a pediatric and/or neonatal parenteral nutrition product and generally provide a carbohydrate formulation as disclosed herein in the first chamber, an amino acid formulation as disclosed herein in a second chamber, and a lipid formulation as disclosed herein in a third chamber of the container.

The multi-chamber container, such as a three-chamber bag, may include vertical chambers. Suitable multichamber containers are disclosed, for example, in U.S. Patent Publication No. 2007/0092579. For example, the multichamber container may be configured as a bag that includes two, three, or more adjacent chambers or compartments. If desired, frangible barriers or openable seals (e.g., peel seals or frangible seals) are used to separate the chambers of the multi-chamber container. Multi-chamber containers may, for example, comprise three chambers for accommodating a lipid emulsion, a carbohydrate formulation, and an amino acid formulation, and may further comprise at least one, in certain embodiments two or three smaller chambers which contain, for example, vitamin formulations and/or trace element formulations. In one specific embodiment, the multi-chamber container of the invention has a first chamber containing the lipid emulsion according to the invention, a second chamber containing an amino acid formulation, a third chamber containing a carbohydrate formulation, and a fourth chamber containing a vitamin formulation. Optionally, it may contain a fifth chamber containing a trace element formulation.

The openable seals of said multi-chamber containers permit formulations to be separately stored and admixed/reconstituted just prior to administration, thereby allowing storage in a single container of formulations which should not be stored as an admixture for an extended period of time. Opening of the seals allows communication between the chambers and mixing of the contents of the respective chambers. The outside seals of the multi-chamber container are strong seals that do not open under the fluid pressure supplied to open the weaker peel seals or frangible seals between the chambers. In some embodiments, the openable seals of the multi-chamber container may be designed to allow for the admixing or reconstitution of only selected chambers of the multi-chamber container, for example, the admixing of the lipid emulsion with the vitamin chamber and the amino acid chamber, if so desired.

The multi-chamber container may be provided with instructions explaining a desired order with which to open the peel seals, so that constituent fluids are mixed in a desired order. The unsealing strengths of the two or more peel seals may be varied to promote the opening of the seals in the desired order. For example, the unsealing strength of the peel seal to be opened first may be ⅓ to ½ of the unsealing strength required to open the peel seal to be opened second.

Where a lipid emulsion according to the invention is included into a multi-chamber bag, the concentration of the respective components in the lipid emulsion such as ARA, DHA, EPA, choline, linolenic acid etc. may be adapted as needed to arrive at desired final concentrations in the reconstituted solution. However, lipid emulsions in a multichamber container may also comprise concentrations within the ranges as disclosed before.

As used herein, “reconstituted solution” refers to a solution for parenteral administration, which is generated by admixing the content of the chambers of a multichamber container before use.

As mentioned above, typical components of a multi-chamber container for providing formulations for parenteral nutrition are amino acid and/or carbohydrate formulations. The amino acid formulations include a sterile, aqueous solution of one or more amino acids and one or more electrolytes. Typically, amino acid formulations include about 2 g to about 10 grams of amino acids per 100 mL of amino acid formulation, such as about 3 grams to about 9 grams and/or about 5 grams to about 7 grams per 100 mL of amino acid formulation. Typical amino acids which are included into amino acid formulations are, for example, isoleucine, leucine, valine, lysine, methionine, phenylalanine, threonine, tryptophan, arginine, histidine, alanine, aspartic acid, cysteine, glutamic acid, glycine, proline, serine, tyrosine, ornithine, and taurine. Further, the content of tyrosine can be increased by adding, for example, a glycyltyrosine dipeptide or acetyl-tyrosine (Ac-Tyr). Typically, however, the glycyl-tyrosine dipeptide has improved pharmacokinetics compared to Ac-Tyr, which is more rapidly eliminated by the kidney, resulting in diminished release of tyrosine in the blood.

The amino acid formulation may further include electrolytes such as sodium, potassium, calcium, magnesium, and/or phosphate ions. For example, the amino acid formulation can include from about 0.1 mmol to about 10 mmol of sodium (e.g., about 3.75 mmol to about 10 mmol of sodium), from about 0.1 mmol to about 10 mmol of potassium (e.g., about 3.75 mmol to about 6.90 mmol of potassium), from about 0.05 mmol to about 1.0 mmol of magnesium (e.g., about 0.05 mmol to about 0.11 mmol and/or about 0.38 mmol to about 0.65 mmol of magnesium), from about 0.1 mmol to about 10 mmol of calcium (e.g., about 1.13 mmol to about 5.10 mmol of calcium), from about 0.1 mmol to about 10 mmol of phosphate (e.g., about 0.94 mmol to about 5.10 mmol of phosphate) and not more than 10 mmol of chloride (e.g., not more than 5.6 mmol of chloride) per 100 mL of amino acid formulation. When calcium and phosphorus are present together in the same heat-sterilized solution, insoluble calcium phosphate precipitation can occur. Using an organic salt of phosphorus such as sodium glycerophosphate 5·H₂O or calcium glycerophosphate, calcium and phosphate amounts may be increased without solubility issues and without providing excess sodium or chloride. In the amino acid formulation, sodium may be provided in the form of sodium chloride, calcium may be provided in the form of calcium chloride 2·H₂O or calcium gluconate, magnesium may be provided in the form of magnesium acetate 4·H₂O or magnesium chloride, and potassium may be provided in the form of potassium acetate.

The carbohydrate formulations provide a supply of calories, typically in the form of glucose. In particular, the carbohydrate formulation provides an amount of carbohydrate sufficient to avoid adverse effects such as hyperglycemia that has been observed in patients receiving parenteral nutrition. Typically, the carbohydrate formulation includes about 20 to 50 grams of glucose per 100 mL of carbohydrate formulation.

The lipid emulsions of the invention can be prepared according to generally known processes (see, for example, Hippalgaonkar et al., 2010, AAPS PharmSciTech, 11 (4): 1526-1540). Generally, water soluble and oil-soluble ingredients are dissolved in the aqueous phase and oil phase, respectively. Accordingly, GPC is dissolved in the aqueous phase. Emulsifiers, such as phosphatides, can be dispersed in either the oil or aqueous phase. Both phases are adequately heated and stirred to disperse or dissolve the ingredients. The lipid phase is then generally added to the aqueous phase under controlled temperature and agitation (using high-shear mixers) to form a homogenously dispersed coarse or pre-emulsion. Pre-emulsions with a droplet size smaller than 20 μm generally produces unimodal and physically stable fine emulsions. The pre-emulsion is then homogenized (using a microfluidizer or a high-pressure homogenizer) at optimized pressure, temperature, and number of cycles to further reduce the droplet size and form a fine emulsion. Factors such as type and concentration of oil phase and surfactants, operating temperature, pressure, number of cycles, etc. can influence the mean droplet size during high-pressure homogenization and microfluidization. Throughout the shelf-life of an emulsion, the mean droplet size and PFAT5 (volume-weighted percentage of fat globules ≥5 μm) of an injectable fine emulsion should be ≤500 nm and ≤0.05%, respectively. The pH of the resulting fine emulsion is then adjusted to the desired value and the emulsion is filtered through 1-5 μm filters. The fine emulsions are then transferred into suitable containers. Plastic containers which are permeable to oxygen and/or contain oil-soluble plasticizers and usually avoided. The entire process (filtration/coarse and fine emulsion preparation) is usually carried out under nitrogen atmosphere whenever possible and especially in cases where the excipients and specific components of the lipid emulsion are sensitive to oxidation. Sterilization of the lipid formulations can be achieved by terminal heat sterilization or by aseptic filtration. Terminal sterilization generally provides greater assurance of sterility of the final product. However, if the components of the emulsions are heat labile, sterile filtration can be used. Sterilization by filtration requires the emulsion droplet size to be below 200 nm. Alternatively, aseptic processing may be employed. However, this process is relatively equipment and labor intensive and requires additional process validation data and justification during regulatory submissions.

Accordingly, lipid emulsions according to the invention can be prepared by the following steps comprising
(a) Separately heating up the oil phase and the aqueous phase to a temperature of from about 70° C. to about 80° C. under agitation;
(b) Adding ARA, DHA and optionally EPA and/or linolenic acid to the oil phase;
(c) Optionally adding choline and α-tocopherol to the aqueous phase;
(d) Preparing the pre-emulsion by mixing the oil phase and the aqueous phase;
(e) Homogenizing the pre-emulsion at a temperature of from about 40° C. to 80° C. under pressure;
(f) Optionally adding water to adjust the required volume and concentrations;
(g) Optionally adjusting the pH to a range of from about 6.0 to 9.0; and
(h) Optionally sterilizing the lipid emulsion.

Sterilization can be done by methods known in the art, such as, for example, by heat. Usually, steps (a) through (d) will be performed in the presence of an inert gas, such as N₂, for avoiding any oxidation reactions. The pressure used for homogenization of the pre-emulsion can vary over a broad range. Generally, it will be in a range of from 100 to 1300 bar, from 200 to 1000 bar, from 300 to 800 bar, or from 400 to 1100 bar.

As discussed before, the invention relates to lipid emulsions and compositions comprising same for avoiding and/or treating liver damage, inflammation and/or fatty acid imbalance including insufficient or inadequate development of certain tissues such as liver, lung, retina and brain, especially lung and retina, especially in pediatric patients including pre-term and extremely pre-term babies.

### EXAMPLES

### Example 1: Preparation of 20% Lipid Emulsions

Lipid emulsions are prepared from raw materials based on what is shown in Table I.

Where choline is added to the lipid emulsion, such choline can be added as choline chloride or GPC. Choline chloride (2-Hydroxyethyl) trimethylammonium chloride), Pharmaceutical Secondary Standard, can be purchased from Sigma-Aldrich (Merck KGAA, Germany). Glycerophosphocholine, prepared from egg phosphatide by saponification, is provided by Lipoid GmbH, Germany. DHA (from algae, Schizochytrium sp.) and ARA (from fungi, Mortierella alpina) can be purchased from BASF AG, Germany as a blend of DHA and ARA. Vitamin E (all-rac-α-tocopherol) can be purchased from ADM (IL, U.S.A.). Raw materials as used for preparing exemplary lipid emulsions such as provided for carrying out the animal study (Example 2) are shown in Table I.

TABLE I
Raw materials used for the preparation of an “NGF” lipid
emulsion according to the invention. Formulation “NGF 2”
was used for carrying out the animal study that is further
described in the following Examples and Tables.

| | NGF 1 | NGF 2 | NGF 3 |
|---|---|---|---|
| | Content | Content | Content |
| | per L | per L | per L |
| Materials | 20% LE | 20% LE | 20% LE |
| Olive oil | 67.0 g* | 112.5 g* | 93.4 g* |
| Soybean oil | 44.9 g* | 43.4 g* | 40.3 g* |
| Blend of DHA | 88.1 g | 44.1 g | — |
| and ARA (1:1) | | | |
| Blend of DHA | — | — | 66.2 g |
| and ARA (1:2) | | | |
| GPC | — | 4 g | 4 g |
| Egg phosphatide | 12 g | 12 g | 12 g |
| Tocopherol | 0.16 g | 0.17 g | 0.17 g |
| Glycerol | 22.5 g | 22.5 g | 22.5 g |
| Sodium oleate | 0.3 g | 0.3 g | 0.3 g |
| Sodium hydroxide | Q.S. pH | Q.S. pH | Q.S. pH |
| Water for injection | Q.S. | Q.S. | Q.S. |
| Nitrogen | | | |

*Oil quantities are adapted according to their content of EFA of the respective oil to provide for a linoleic acid (LA) concentration as intended

Where DHA and ARA are derived from algae, the respective oils used alone or in the form of a blend (see Table I) may contain low amounts of phytosterols which are then added to the olive and soybean oils. These low amounts of phytosterols can generally be absorbed by the phytosterol-reduced olive and soybean oils without deviating from the desired phytosterol levels according to the invention. Desmosterol may be present or added in higher quantities when using algae as a source for DHA and ARA, as shown in Table II. However, the phytosterol level remains low, even if the total phytosterol level increases due to the addition of desmosterol. Desmosterol, as mentioned before, does not seem to negatively impact the effects of the lipid emulsions of the invention.

TABLE II
Phytosterol and desmosterol content in lipid
emulsions according to the invention

| Content (mg/L) | NGF 1 | NGF 2 | NGF 3 |
|---|---|---|---|
| Phytosterol | 139 | 104 | 120 |
| Desmosterol | 262 | 131 | 235 |
| Total phytosterol | 401 | 235 | 355 |
| and desmosterol | | | |

For comparison, SMOFlipid which was used in the animal study has been reported in Osowska et al., 2022, Potential for Omega-3 Fatty Acids to Protect against the Adverse Effect of Phytosterols: Comparing Laboratory Outcomes in Adult Patients on Home Parenteral Nutrition Including Different Lipid Emulsions, Biology 11 (12): 1699 to contain 186.3 mg/L (18.63 mg/dL) plant sterols.

The NGF lipid emulsions according to the invention are prepared according to known methods. Protection against light and oxygen should be maintained throughout the process as e.g., DHA and ARA are prone to oxidation. In a first step, raw phases are heated up. In case of the oil phase, soybean and olive oil as well as DHA and ARA oils are combined before starting the procedure. Then egg phosphatide and vitamin E are added, and the mixture is heated to about 70° C.-80° C.° C. under N₂ protection. The aqueous phase is prepared by mixing glycerol, sodium oleate and water under heating to about 70° C.-80° C. under N₂ protection. The respective choline derivative can be added where applicable. In a second step, the pre-emulsion of above is prepared by adding the oily phase to the aqueous phase and transferring the mix to an inline disperser. In a third step the pre-emulsion is homogenized by passing it through a high-pressure homogenizer. The emulsion is then adjusted to the required volume and cooled down. The pH is adjusted to the desired range. Dissolved oxygen should remain at low levels. The emulsion is filtered and transferred into the container bags, which are then overpouched, optionally together with an oxygen absorber. The lipid emulsion is then autoclaved at 121° C. The composition should be milky white upon visual inspection which indicates emulsion stability (no dephasing, coalescence etc.).

The stability of the lipid emulsions is evaluated by testing physicochemical, assay and impurity parameters at time zero and throughout storage. Also, at time zero and at the end of the animal study, the compatibility of the lipid emulsions with other components of the parenteral nutrition (i.e., glucose, amino acids, vitamins) is confirmed. PFAT5 analysis is used to determine the percentage of fat residing in globules larger than 5 μm (PFAT5) for a given lipid injectable emulsion and which is not to exceed 0.05%. Mean globule size (MGS) is determined with a Coulter counter and must not exceed 500 nm according to USP requirements and preferably should be below 350 nm in the present emulsions. In addition, particulate contamination is evaluated. Particulate matter larger than 10 μm should not exceed 12 particles/ml in the experimental emulsion.

Total oil in the lipid emulsion can be determined by high performance liquid chromatography (HPLC), and the fatty acid composition is determined by gas chromatography as mentioned before. The hydrolysis of the oils and the formation of free fatty acids can be evaluated with the help of Ultraviolet visible spectrophotometers. Tocopherol can be determined using Ultra performance liquid chromatography with a fluorescence detector (FLR). The method can be used to determine α-tocopherol and the total tocopherol content. GPC can be determined using Ultra performance liquid chromatography with an evaporative light scattering detector (ELSD). Peroxide generation can be monitored using Ultraviolet visible spectrophotometers. The degradation of fatty acids due to oxidation can result in the generation of aldehydes. When diluted in isooctane, aldehydes react with 4-methoxyaniline (p-anisidine) in presence of acetic acid and colored derivatives are formed that have a maximum absorbance at 350 nm. This provides for the Anisidine Value of an oil or lipid emulsion. Finally, phytosterols and desmosterol present in e.g., soybean oil, olive oil, as well as DHA and ARA oil can be determined by gas chromatography using a flame ionization detector. The method is able to detect and quantify desmosterol, stigmasterol, campesterol, beta-sitosterol, sitostanol, lanosterol, ergosterol, lathosterol, brassicasterol, cholesterol and squalene.

### Example 2: Animal Model

Animal studies were performed for determining the most effective combinations of various PUFAs including EPA, DHA, ARA as well as their optimal concentrations and ratios for use in parenteral nutrition solutions, specifically in lipid emulsions, for avoiding and/or treating and ameliorating liver damage, inflammation and fatty acid imbalance including impaired or insufficient development of certain tissues relevant especially in pediatric patients and especially in pre-term and extremely pre-term babies, such as lung and retina. The study further served to identify any potential metabolic interactions between ARA, DHA and EPA as well as choline, linolenic acid and α-tocopherol regarding the above issues which are of concern in said pediatric patients. The studies further served to evaluate the influence the presence and amount of phytosterol on said conditions and their potential metabolic interaction with the other components of the lipid emulsion that were found to be of relevance, specifically of DHA and ARA.

The effects of ARA, DHA, and EPA in the disclosed concentration ranges and ratios, optionally in combination with choline, specifically GPC, linolenic acid, α-tocopherol and/or phytosterols were investigated in an animal model, wherein parenteral nutrition (PN)-treated neonatal piglets had jugular venous catheter insertion, nutrition, and care according to what is described in Josephson J, Turner J M, Field C J et al., 2015, Parenteral soy oil and fish oil emulsions: impact of dose restriction on bile flow and brain size of parenteral nutrition-fed neonatal piglets. JPEN J Parenter Enteral Nutr. 39:677-687 for 14 days. Baseline samples are taken at Day 0 which is also the day of jugular catheter implantation. Total parenteral nutrition is provided on Day 1-Day 14. Piglets received perioperative antibiotics. Bile flow is tested on Day 14 and post treatment samples are collected.

The study was used to compare two conventional, commercially available lipid emulsions, Intralipid (Fresenius Kabi) and SMOFlipid (Fresenius Kabi), with new lipid emulsions according to the invention, herein also referred to as “NGF” emulsions, wherein various parameters were monitored (bile flow) and post treatment samples were taken to establish the impact of the optimized formulation according to the invention on liver damage and fatty acid profile as well as certain relevant tissues such as retina (eye), brain, lung, liver and intestine (Table III). Examples for specific NGF emulsions (NGF1, NGF2, NGF3) are shown in Tables I and II. “NGF 2” was used in the animal study described herein. The normal range for all parameters reviewed in the study was determined by comparison with equivalent aged sow-reared piglets. Nine (9) piglets were allocated to Intralipid of which seven (7) completed the trial, ten (10) were allocated to SMOFlipid of which seven (7) completed the trial, and eleven (11) were allocated to the NGF 2 formulation according to the invention (“NGF 2”) of which eight (8) completed the trial. Eight (8) sow-reared piglets served as controls.

TABLE III
Composition of tested lipid emulsions according to the invention
and of commercially available controls SMOF-lipid and Intralipid.
NGF 2 was used in the animal study (Example 2)

| | Controls | | Invention | | | |
|---|---|---|---|---|---|---|
| | Intra- | SMOF- | NGF 1 | NGF 1 | | |
| Compound | lipid | lipid | (a) | (b) | NGF 2 | NGF 3 |
| DHA (g/L) | 0.12 | 3, 5 to | 20 | 20 | 10 | 10 |
| | | 4, 4 | | | | |
| EPA (g/L) | <0.02 | 4, 8 to | 1.0 | 1.0 | 0.5 | 0.5 |
| | | 6, 1 | | | | |
| DHA/EPA | NA | ~1/1.4 | 20/1 | 20/1 | 20/1 | 20/1 |
| Ratio | | | | | | |
| ARA (g/L) | 0.2 | 0.9 | 21 | 21 | 10.5 | 21 |
| DHA/ARA | NA | ~5:1 | 1/1 | 1/1 | 1/1 | 1/2 |
| Ratio | | | | | | |
| LA (g/L) | 36 | 35 | 39 | 39 | 39 | 39 |
| ω-6/ω-3 Ratio | 9:1 | 2.5:1 | 2.3/1 | 2.3/1 | 3.3/1 | 3.9/1 |
| α-Tocopherol | 28-36 | 200 | 200 | 200 | 200 | 200 |
| (mg/L) | | | | | | |
| Phytosterols | 201 mg/L | 163 mg/L | 139 | 139 | 104 | 120 |
| in olive oil | | | | | | |
| and soybean | | | | | | |
| oil | | | | | | |
| GPC (g/L) | — | — | 0 | 4 | 4 | 4 |

The dose of lipid was 10 g/kg/d in the PN-treated piglets. As piglets grow at 5 times the rate of human infants, the doses translate to approximately 2 g/kg/d for infants.

In the study, neonatal piglets totally fed by means of parenteral nutrition were used to compare a lipid emulsion according to the invention (“NGF 2”) with standard commercial lipid emulsions (Intralipid and SMOF) regarding their impact on the following key outcomes: 1. Blood and Tissue fatty acid composition, including plasma, brain, liver, retina and lung. 2. Liver chemistry and histology, bile acid excretion, bile acid composition and metabolism. 2-3-day old Duroc cross Landrace/Large White piglets were block randomized to four treatment groups as described above. Piglets underwent general anesthesia and insertion of a jugular venous catheter as per standard protocol. Isonitrogenous and isocaloric parenteral nutrition (260 kcal/kg/d and 16 g/kg/d amino acids) commenced immediately postoperatively using standardized PN admixtures, including amino acid, glucose, electrolytes, vitamins and trace elements. Lipids were added immediately to the standardized PN prior to infusion to piglets. PN was provided at a final infusion rate of 13.5 ml/kg/hr. Target nutrient intakes were 260 Kcal/kg/d total energy and 16 g/kg/d amino acids, prior to terminal laparotomy, measurement of bile flow, euthanasia and collection of tissue samples. Piglets will were weighed on a daily basis while on trial and fluid balance was monitored. Parenteral antibiotics were given per protocol to all piglets immediately post-operatively to prevent line- and/or surgery-related sepsis. Enteral nutrition was not provided.

### Example 3: Anthropometric Data (Median/IQR)

The weight of the piglets was determined at DO and D14 and is shown in Table IV. In addition, the weight of the liver, small bowel and brain were determined once the study was concluded (D14). There were no major differences in the body weight. Liver weight was higher in all test groups versus the control, whereas the weight of the small bowel was lower in all test groups compared to the control group and appeared to be especially low in the Intralipid group. Brain weight was also slightly lower on the test groups and appeared lowest in the SMOFlipid group, however, with only small differences versus Intralipid group and NGF 2 group.

TABLE IV
Anthropometric data (median/IQR) for test and control groups

| | Intralipid | SMOFlipid | NGF 2 | Control | |
|---|---|---|---|---|---|
| | (n = 7) | (n = 7) | (n = 8) | (n = 8) | p value |
| Weight D0 | 2.2 | 2.5 | 2.3 | — | 0.24 |
| (kg) | (0.3) | (0.4) | (0.4) | | |
| Weight | 5.1 | 5.7 | 5.2 | 7.0 | <0.001 |
| D14 (kg) | (0.5) ^{a} | (1.0) ^{a} | (0.4) ^{a} | (1.2) ^{b} | |
| Liver | 234.9 | 268.2 | 257.1 | 185.1 | <0.001 |
| weight | (42.8) ^{a} | (20.7) ^{a} | (40.1) ^{a} | (31.1) ^{b} | |
| (g) | | | | | |
| Small | 80.6 | 110.0 | 102.3 | 225.0 | <0.001 |
| bowel | (24.9) ^{a} | (23.4) ^{b} | (12.5) ^{b} | (16.0) ^{c} | |
| weight | | | | | |
| (g) | | | | | |
| Brain | 40.3 | 37.6 | 38.9 | 42.9 | 0.017 |
| weight | (3.4) ^{a} | (5.9) ^{a} | (3.8) ^{a} | (0.7) ^{b} | |
| (g) | (n = 4) | (n = 5) | | (n = 5) | |

### Example 4: Plasma and Tissue Fatty Acid Analysis

At trial completion, Day 14, whole blood is collected in sodium citrate and centrifuged. The plasma (platelet) portion is removed and further microcentrifuged. Supernatant is discarded, leaving the isolated platelet plug that is immediately frozen and stored at −80° C. prior to further analysis. Plasma samples are collected for fatty acid analysis in phospholipids. Separation of phospholipids from other major lipid classes and methylation can be performed by thin-layer chromatography (Field et al., 1988, Dietary fat and the diabetic state alter insulin binding and the fatty acyl composition of the adipocyte plasma membrane. Biochem. J., 253 (2): 417-424.) In case of liver, fatty acids were analyzed from both phospholipids and triglycerides (TG). The phospholipid band is visualized and scraped, and 10 μg C17:0 (100 μL of 10 mg/100 mL) triglyceride standard is added to the silica prior to methylation with BF₃ (Sigma-Aldrich, Mississauga, ON, Canada). Fatty acids are separated and identified by gas liquid chromatography (Agilent GC model 7890a; Agilent, Wilmington, DE, USA) using a 100-m CP-Sil 88 fused capillary column (Varian Instruments, Mississauga, ON, Canada) as previously described (Cruz-Hernandez et al., 2004, Methods for analysis of conjugated linoleic acids and trans-18:1 isomers in dairy fats by using a combination of gas chromatography, silver-ion thin-layer chromatography/gas chromatography, and silver-ion liquid chromatography. J AOAC Int.; 87 (2): 545-562).

For fatty acid analysis of tissue, the respective tissue (100 mg) is ground for 1 minute in 500 μL of phosphate-buffered saline (PBS) solution and extracted and methylated using the method described above. The fatty acid composition of the tissue fat emulsion is analyzed by gas chromatography (see above) and expressed as a percentage of total fatty acids.

TABLE V
% Polyunsaturated fatty acids in serum phospholipid
(mean/SD)

| | Intralipid | SMOFlipid | NGF 2 | Control | |
|---|---|---|---|---|---|
| PUFA | (n = 7) | (n = 7) | (n = 8) | (n = 8) | P Value |
| C18:1n9 | 14.0 | 16.7 | 17.0 | 12.5 | <0.001 |
| | (2.5) ab | (1.2) ab | (2.2) b | (1.4) a | |
| C18:1n7 | 1.6 | 1.8 | 2.1 | 2.4 | <0.001 |
| | (0.3) a | (0.1) ab | (0.4) bc | (0.3) c | |
| C18:2n6 | 14.3 | 10.0 | 6.3 | 20.6 | <0.001 |
| (LA) | (5.6) a | (1.6) ab | (1.7) b | (1.4) c | |
| C18:3n3 | 0.4 | 0.3 | 0.4 | 0.9 | 0.007 |
| (ALA) | (0.06) a | (0.04) a | (0.1) a | (0.2) b | |
| C18:3n6 | 0.2 | 0.1 | 0.2 | 0.06 | <0.001 |
| | (0.05) a | (0.1) ab | (0.1) a | (0.02) b | |
| C20:2n6 | 0.6 | 0.4 | 0.4 | 0.2 | <0.001 |
| | (0.06) a | (0.05) b | (0.1) b | (0.03) b | |
| C20:3n6 | 2.5 | 2.2 | 1.3 | 1.1 | <0.001 |
| | (0.9) a | (0.4) a | (0.3) b | (0.1) b | |
| C20:4n6 | 7.5 | 5.4 | 9.8 | 7.7 | <0.001 |
| (AA) | (2.2) ab | (0.9) b | (1.8) c | (0.9) ac | |
| C20:4n3 | 0.2 | 0.2 | 0.1 | 0.1 | <0.001 |
| | (0.1) a | (0.04) a | (0.05) a | (0.01) b | |
| C20:5n3 | 0.1 | 0.8 | 0.3 | 0.3 | 0.003 |
| (EPA) | (0.03) a | (0.3) b | (0.6) a | (0.1) a | |
| C22:4n6 | 0.4 | 0.3 | 0.5 | 0.3 | 0.22 |
| | (0.1) | (0.1) | (0.4) | (0.03) | |
| C22:5n6 | 0.3 | 0.3 | 0.4 | 0.2 | 0.005 |
| | (0.09) ab | (0.07) a | (0.2) a | (0.03) b | |
| C22:5n3 | 0.5 | 1.1 | 0.3 | 1.5 | <0.001 |
| | (0.4) a | (0.3) b | (0.1) a | (0.2) b | |
| C22:6n3 | 1.7 | 2.8 | 2.4 | 2.2 | 0.11 |
| (DHA) | (0.8) | (0.9) | (0.9) | (0.4) | |
| C24:1n9 | 0.6 | 1.4 | 0.8 | 0.8 | <0.001 |
| | (0.3) a | (0.3) b | (0.2) a | (0.1) a | |

As can be seen for arachidonic acid, its prevalence is clearly increased for those animals that received NGF 2 in comparison to the commercial products and even relative to the Control (sow-reared piglets).

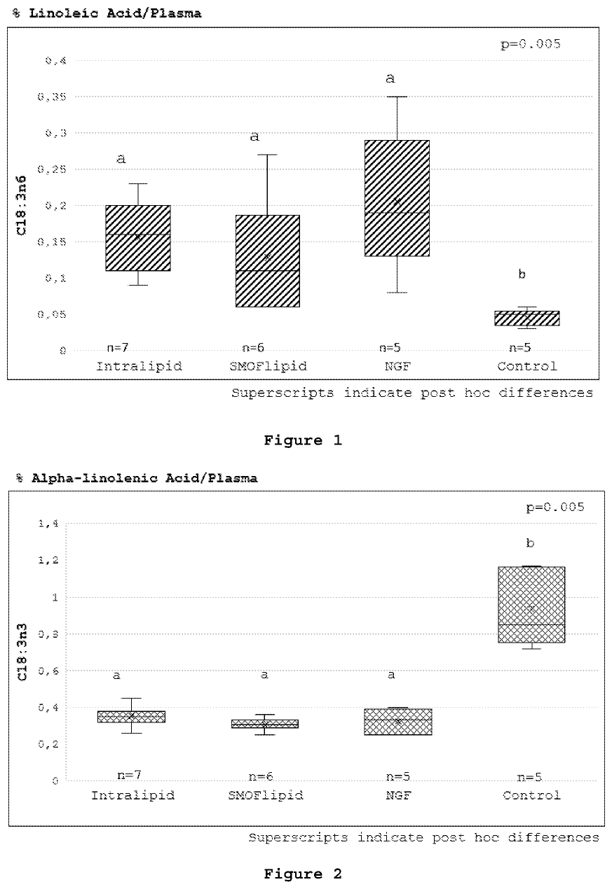

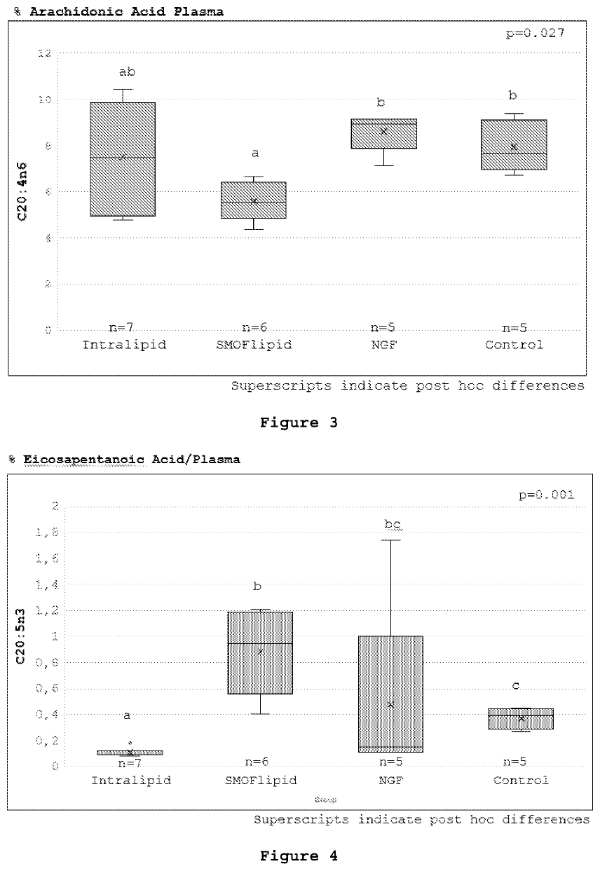

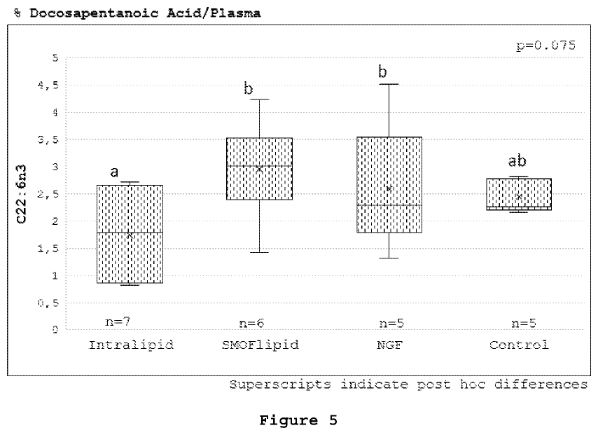

The results obtained for plasma levels of linoleic acid, alpha-linolenic acid, arachidonic acid, eicosapentaenoic acid, and docosapentaenoic acid are also provided in FIGS. 1 to 5. The plasma levels obtained with piglets parenterally fed with NGF 2 according to the invention show high levels of desirable fatty acids that are generally as high as or even higher than those obtained with comparative products. Levels of linoleic acid and arachidonic acid are significantly higher than SMOFlipid levels. For ARA, the obtained levels are as high as the levels reached in the Control group of sow-fed piglets. Despite the relatively low EPA content of NGF 2, the plasma levels were found to be about equivalent to SMOFlipid that has a higher EPA content, and to the Control. Only Intralipid was found to be even below the EPA serum level obtained with NGF 2. The DHA plasma level obtained with NGF 2 was also found to be equivalent to the Control and about equivalent to SMOFlipid.

The results of the tissue fatty acid analysis are shown in Tables VI to X, with some unexpected results underlining the surprising benefits of the lipid emulsions disclosed herein. Data were analyzed as ANOVA when 98% or more parametric distribution was found. Otherwise, data were analyzed according to Kruskal-Wallis if not parametric or key data was not parametric. Superscripts indicate post-hoc differences by Tukey or Mann Whitney according to data distribution.

TABLE VI
% Polyunsaturated fatty acid in liver phospholipid
(mean/SD)

| | | SMOF- | | | |
|---|---|---|---|---|---|
| | Intralipid | lipid | NGF 2 | Control | P value |
| | (n = 7) | (n = 7) | (n = 8) | (n = 8) | ANOVA |
| C18:1n9 | 11.6 | 14.9 | 13.1 | 8.9 | <0.001 |
| | (1.2) | (1.4) | (1.2) | (0.9) | |
| C18:1n7 | 1.4 | 1.8 | 2.0 | 2.3 | <0.001 |
| | (0.2) | (0.1) | (0.2) | (0.2) | |
| C18:2n6 | 19.0 | 13.1 | 6.5 | 13.0 | <0.001 |
| (LA) | (2.9) | (0.5) | (0.7) | (0.7) | |
| C18:3n3 | 2.4 | 0.1 | 0.1 | 0.1 | <0.001 |
| (ALA) | (1.2) | (0.02) | (0.02) | (0.02) | |
| C18:3n6 | 0.4 | 0.3 | 0.2 | 0.4 | <0.001 |
| | (0.1) | (0.04) | (0.03) | (0.07) | |
| C20:2n6 | 0.9 | 0.3 | 0.3 | 0.2 | <0.001 |
| | (0.2) | (0.06) | (0.04) | (0.02) | |
| C20:3n6 | 3.4 | 2.5 | 1.8 | 1.0 | 0.003 |
| | (2.2) | (0.7) | (0.2) | (0.2) | |
| C20:4n6 | 14.2 | 10.2 | 22.5 | 15.2 | <0.001 |
| (AA) | (1.7) | (0.5) | (0.8) | (1.2) | |
| C20:4n3 | 0.1 | 1.7 | 0.2 | 0.3 | 0.15 |
| | (0.04) | (0.4) | (0.1) | (0.1) | |
| C20:5n3 | 0.2 | 1.5 | 0.5 | 0.3 | <0.001 |
| (EPA) | (0.2) | (0.5) | (0.2) | (0.05) | |
| C22:4n6 | 0.6 | 0.3 | 0.6 | 0.6 | <0.001 |
| | (0.08) | (0.08) | (0.05) | (0.05) | |
| C22:5n6 | 0.4 | 0.3 | 0.4 | 0.4 | <0.001 |
| | (0.1) | (0.04) | (0.05) | (0.1) | |
| C22:5n3 | 0.8 | 2.4 | 0.6 | 2.2 | <0.001 |
| | (0.3) | (0.5) | (0.1) | (0.3) | |
| C22:6n3 | 4.0 | 7.6 | 7.8 | 5.5 | <0.001 |
| (DHA) | (0.3) | (0.7) | (1.0) | (0.7) | |
| C24:1n9 | 0.2 | 0.4 | 0.4 | 0.9 | <0.001 |
| | (0.02) | (0.09) | (0.08) | (0.3) | |

The results shown in Table VI indicate that especially the prevalence (deposition) of arachidonic acid (AA) is surprisingly high in the liver of animals parenterally fed with the NGF 2 formulation according to the invention. This finding could be confirmed when looking at the polyunsaturated fatty acids in triglyceride form, see Table VII. Also, in this case ARA deposition is clearly increased.

TABLE VII
% Polyunsaturated fatty acid in liver triglyceride
(mean/SD; median/IQR)

| | Intralipid | SMOFlipid | NGF 2 | Control | P value |
|---|---|---|---|---|---|
| | (n = 7) | (n = 7) | (n = 8) | (n = 8) | ANOVA |
| C18:1n9 | 21.5 (4.9) | 27.3 (2.3) | 27.2 (2.8) | 26.4 (3.0) | 0.035 |
| | 21.6 (8.4) ^{a} | 27.6 (5.1) ^{b} | 27.7 (5.2) ^{b} | 26.9 (5.8) ^{b} | |
| C18:1n7 | 1.6 (0.3) | 2.2 (0.3) | 2.4 (0.3) | 2.9 (0.2) | <0.001 |
| | 1.6 (0.6) ^{a} | 2.2 (0.4) ^{b} | 2.5 (0.5) ^{b} | 2.9 (0.4) ^{c} | |
| C18:2n6 | 33.7 (5.9) | 17.5 (1.2) | 14.5 (1.4) | 12.2 (0.9) | <0.001 |
| (LA) | 36.9(11.9) ^{a} | 17.7 (2.5) ^{b} | 14.5 (2.5) ^{c} | 11.8 (1.8) ^{d} | |
| C18:3n3 | 2.4 1.2) | 1.4 (0.2) | 1.3 (0.3) | 1.5 (0.1) | 0.043 |
| (ALA) | 2.0 (2.5) ^{a} | 1.4 (0.3) ^{b} | 1.3 (0.6) ^{b} | 1.5 (0.3) ^{ab} | |
| C18:3n6 | 1.1 (0.2) | 0.5 (0.1) | 0.9 (0.2) | 0.4 (0.2) | <0.001 |
| | 1.0 (0.4) ^{a} | 0.4 (0.1) ^{b} | 1.0 (0.4) ^{c} | 0.4 (0.3) ^{d} | |
| C20:2n6 | 1.1 (0.3) | 0.6 (0.2) | 0.5 (0.1) | 0.4 (0.1) | <0.001 |
| | 1.0 (0.8) ^{a} | 0.6 (0.3) ^{b} | 0.5 (0.3) ^{b} | 0.3 0.1) ^{c} | |
| C20:3n6 | 1.8 (1.1) | 1.0 (0.3) | 1.2 (0.2) | 0.3 (0.1) | 0.001 |
| | 2.1 (2.2) ^{a} | 0.8 (0.5) ^{a} | 1.2 (0.3) ^{a} | 0.3 (0.2) ^{b} | |
| C20:4n6 | 2.9 (0.7) | 2.3 (0.4) | 7.8 (1.4) | 4.0 (0.9) | <0.001 |
| (AA) | 3.1 (0.9) ^{a} | 2.4 (0.6) ^{a} | 7.9 (2.1) ^{b} | 4.1 (1.6) ^{b} | |
| C20:4n3 | 0.2 (0.1) | 0.3 (0.1) | 0.3 (0.1) | 0.2 (0.1) | 0.026 |
| | 0.2 (0.2) ^{a} | 0.3 (0.1) ^{b} | 0.2 (0.1) ^{b} | 0.2 (0.1) ^{a} | |
| C20:5n3 | 0.2 (0.2) | 1.5 (0.5) | 0.6 (0.2) | 0.3 (0.1) | <0.001 |
| (EPA) | 0.2 (0.04) ^{a} | 1.2 (0.9) ^{b} | 0.6 (0.4) ^{c} | 0.3 (0.2) ^{d} | |
| C22:4n6 | 0.6 (0.4) | 0.3 (0.06) | 1.2 (1.1) | 0.4 (0.1) | 0.52 |
| | 0.4 (0.9) | 0.3 (0.1) | 1.1 (2.2) | 0.3 (0.2) | |
| C22:5n6 | 0.3 (0.2) | 0.4 (0.2) | 1.1 (0.4) | 0.2 (0.1) | <0.001 |
| | 0.4 (0.4) ^{a} | 0.3 (0.3) ^{a} | 0.9 (0.8) ^{b} | 0.2 (0.2) ^{a} | |
| C22:5n3 | 0.4 (0.1) | 3.5 (1.6) | 1.5 (0.4) | 0.5 (0.1) | <0.001 |
| | 0.5 (0.2) ^{a} | 3.0 (3.4) ^{b} | 1.5 (0.8) ^{c} | 0.5 (0.1) ^{a} | |
| C22:6n3 | 1.1 (0.5) | 5.3 (3.0) | 8.2 (3.2) | 0.7 (0.2) | <0.001 |
| (DHA) | 1.4 (0.8) ^{a} | 6.0 (5.8) ^{b} | 7.4 (4.5) ^{b} | 0.6 (0.4) ^{a} | |
| C24:1n9 | 0.3 (0.2) | 0.3 (0.1) | 1.1 (0.6) | 0.2 (0.1) | 0.004 |
| | 0.2 (0.4) ^{a} | 0.3 (0.2) ^{a} | 1.1 (1.2) ^{b} | 0.2 (0.2) ^{a} | |

The deposition of fatty acids in the retina is provided for in Table VIII. The same trend of improved ARA deposition is again visible here. Also, the deposition of DHA is better for the NGF 2 group than in the Control group and the comparative study groups.

TABLE VIII
% Polyunsaturated fatty acids in retina phospholipid
(mean/SD; median/IQR)

| | | | | | P value |
|---|---|---|---|---|---|
| | Intralipid | SMOFlipid | NGF 2 | Control | Kruskal |
| | (n = 6) ^{#} | (n = 7) | (n = 8) | (n = 8) | Wallis |
| C18:1n9 | 14.6 (0.7) | 15.4 (0.8) | 14.9 (0.5) | 14.9 (0.6) | 0.24 |
| | 14.4 (1.1) | 15.8 (1.6) | 14.9 (1.0) | 14.8 (0.4) | |
| C18:1n7 | 3.1 (0.3) | 3.3 (0.2) | 3.1 (0.2) | 3.7 (0.3) | 0.002 |
| | 3.1 (0.7) ^{a} | 3.4 (0.2) ^{a} | 3.1 (0.4) ^{a} | 3.7 (0.5) ^{b} | |
| C18:2n6 | 3.6 (0.7) | 1.9 (0.1) | 1.4 (0.2) | 2.6 (0.4) | <0.001 |
| (LA) | 3.4 (0.8) ^{a} | 1.9 (0.2) ^{b} | 1.4 (0.1) ^{c} | 2.6 (0.5) ^{d} | |
| C18:3n3 | 0.3 (0.2) | 0.2 (0.04) | 0.2 (0.03) | 0.2 (0.06) | <0.001 |
| (ALA) | 0.3 (0.2) ^{a} | 0.2 (0.01) ^{ab} | 0.2 (0.04) ^{b} | 0.2 (0.03) ^{a} | |
| C18:3n6 | 0.2 (0.03) | 0.2 (0.02) | 0.2 (0.03) | 0.1 (0.03) | 0.12 |
| | 0.2 (0.05) | 0.2 (0.04) | 0.2 (0.04) | 0.1 (0.04) | |
| C20:2n6 | 0.4 (0.02) | 0.2 (0.02) | 0.2 (0.01) | 0.2 (0.01) | <0.001 |
| | 0.4 (0.03) ^{a} | 0.2 (0.03) ^{b} | 0.2 (0.03) ^{c} | 0.2 (0.02) ^{ab} | |
| C20:3n6 | 0.6 (0.2) | 0.5 (0.1) | 0.4 (0.03) | 0.6 (0.1) | 0.018 |
| | 0.7 (0.2) ^{a} | 0.5 (0.2) ^{a} | 0.4 (0.04) ^{a} | 0.6 (0.1) ^{b} | |
| C20:4n6 | 8.6 (0.7) | 8.6 (0.2) | 9.4 (0.4) | 9.3 (0.3) | 0.002 |
| (AA) | 8.7 (1.0) ^{a} | 8.6 (0.4) ^{a} | 9.4 (0.6) ^{b} | 9.3 (0.6) ^{b} | |
| C20:4n3 | 0.02 (0.01) | 0.02 (0.01) | 0.01 (0.003) | 0.02 (0.01) | 0.035 |
| | 0.02 (0.01) ^{a} | 0.02 (0.01) ^{ab} | 0.01 (0.0) ^{b} | 0.01 (0.01) ^{ab} | |
| C20:5n3 | 0.05 (0.01) | 0.3 (0.05) | 0.1 (0.02) | 0.07 (0.02) | <0.001 |
| (EPA) | 0.06 (0.02) ^{a} | 0.2 (0.1) ^{b} | 0.1 (0.04) ^{c} | 0.07 (0.03) ^{ac} | |
| C22:4n6 | 1.9 (0.3) | 1.3 (0.1) | 1.7 (0.1) | 1.7 (0.3) | 0.003 |
| | 1.9 (0.4) ^{a} | 1.3 (0.1) ^{b} | 1.7 (0.2) ^{c} | 1.8 (0.5) ^{ac} | |
| C22:5n6 | 1.1 (0.3) | 0.7 (0.1) | 0.7 (0.1) | 0.9 (0.2) | 0.014 |
| | 1.1 (0.6) ^{a} | 0.7 (0.1) ^{b} | 0.7 (0.2) ^{b} | 0.9 ( 0.3) ^{ab} | |
| C22:5n3 | 0.6 (0.1) | 1.0 (0.1) | 0.6 (0.05) | 0.8 (0.1) | <0.001 |
| | 0.6 (0.2) ^{a} | 1.0 (0.2) ^{b} | 0.6 (0.03) ^{a} | 0.8 (0.2) ^{c} | |
| C22:6n3 | 12.9 (2.4) | 14.4 (1.1) | 16.4 (1.1) | 12.8 (2.3) | 0.002 |
| (DHA) | 13.4 (3.0) ^{a} | 14.5 (2.0) ^{a} | 16.6 (1.7) ^{b} | 13.3 (2.4) ^{a} | |
| C24:1n9 | 0.05 (0.02) | 0.05 (0.02) | 0.03 (0.005) | 0.06 (0.03) | 0.041 |
| | 0.05 (0.04) ^{a} | 0.05 (0.03) ^{a} | 0.03 (0.01) ^{b} | 0.06 (0.03) ^{a} | |

^{#}One outlier excluded

Table IX provides for the results found in the lung of animals that were parenterally fed with NGF 2 according to the invention. The development of lung tissue is extremely important especially in pre-term and extremely pre-term babies. Also in this tissue, the ARA deposition is clearly improved versus the prior art and even the Control in the NGF 2 group.

TABLE IX
% Polyunsaturated fatty acids in lung phospholipid
(mean/SD)

| | Intralipid | SMOFlipid | NGF 2 | Control | P value |
|---|---|---|---|---|---|
| | (n = 7) | (n = 7) | (n = 8) | (n = 8) | ANOVA |
| C18:1n9 | 15.9 (0.5) ^{ab} | 19.8 (0.6) ^{b} | 20.8 (0.3) ^{b} | 16.2 (0.6) ^{a} | <0.001 |
| C18:1n7 | 1.9 (0.1) ^{a} | 2.3 (0.1) ^{b} | 2.6 (0.1) ^{c} | 2.9 (0.3) ^{d} | <0.001 |
| C18:2n6 | 17.7(2.0) ^{a} | 10.0 (0.5) ^{ab} | 5.9 (0.4) ^{b} | 7.3 (0.5) ^{b} | <0.001 |
| (LA) | | | | | |
| C18:3n3 | 0.9 (0.08) ^{a} | 0.6 (0.06) ^{b} | 0.5 (0.03) ^{b} | 0.5 (0.1) ^{b} | <0.001 |
| (ALA) | | | | | |
| C18:3n6 | 0.1 (0.06) ^{a} | 0.1 (0.02) ^{a} | 0.1 (0.04) ^{a} | 0.08 (0.05) ^{b} | 0.016 |
| C20:2n6 | 1.0 (0.1) ^{a} | 0.4 (0.05) ^{b} | 0.3 (0.02) ^{bc} | 0.3 (0.03) | <0.001 |
| C20:3n6 | 1.8 (0.3) ^{a} | 1.6 (0.1) ^{ab} | 1.4 (0.2) ^{b} | 1.5 (0.1) ^{ab} | 0.008 |
| C20:4n6 | 7.1 (0.7) ^{a} | 5.6 (0.4) ^{b} | 12.2 (0.5) ^{c} | 9.0 (0.8) ^{d} | <0.001 |
| (AA) | | | | | |
| C20:4n3 | 0.03 (0.01) ^{a} | 0.09 (0.01) ^{b} | 0.03 (0.01) ^{a} | 0.04 (0.01) ^{a} | <0.001 |
| C20:5n3 | 0.1 (0.04) ^{a} | 2.5 (0.3) ^{b} | 0.2 (0.04) ^{c} | 0.4 (0.07) ^{c} | <0.001 |
| (EPA) | | | | | |
| C22:4n6 | 1.0 (0.1) ^{a} | 0.5 (0.1) ^{b} | 1.3 (0.1) ^{c} | 1.2 (0.1) ^{ac} | <0.001 |
| C22:5n6 | 0.2 (0.03) ^{a} | 0.2 (0.03) ^{a} | 0.5 (0.07) ^{b} | 0.1 (0.08) ^{a} | <0.001 |
| C22:5n3 | 0.6 (0.1) ^{a} | 2.2 (0.4) ^{b} | 0.5 (0.06) ^{a} | 1.4 (0.2) ^{c} | <0.001 |
| C22:6n3 | 0.6 (0.1) ^{a} | 1.8 (0.4) ^{b} | 3.2 (0.4) ^{c} | 0.6 (0.1) ^{a} | <0.001 |
| (DHA) | | | | | |
| C24:1n9 | 0.9 (0.2) ^{ab} | 1.2 (0.3) ^{bc} | 1.1 (0.3) ^{bc} | 1.4 (0.1) ^{c} | 0.003 |

The trend of improved ARA deposition is also confirmed in the tissue of the jejunum of piglets of the NGF 2 group, where DHA is also well represented, see Table X.

TABLE X
% Polyunsaturated fatty acids in jejunum phospholipid
(mean/SD)

| | Intralipid | SMOFlipid | NGF 2 | Control | P value |
|---|---|---|---|---|---|
| | (n = 7) | (n = 7) | (n = 8) | (n = 8) | ANOVA |
| C18:1n9 | 12.0 (0.5) ^{a} | 14.9 (0.6) ^{b} | 14.9 (1.4) ^{b} | 12.3 (1.1) ^{a} | <0.001 |
| C18:1n7 | 1.4 (0.3) ^{a} | 1.7 (0.2) ^{ab} | 1.9 (0.2) ^{bc} | 2.1 (0.3) ^{c} | <0.001 |
| C18:2n6 | 18.9 (2.1) ^{a} | 13.8 (1.3) ^{b} | 7.5 (1.1) ^{c} | 20.4 (3.6) ^{a} | <0.001 |
| (LA) | | | | | |
| C18:3n3 | 0.7 (0.2) ^{a} | 0.4 (0.07) ^{b} | 0.4 (0.1) ^{b} | 1.2 (0.2) ^{c} | <0.001 |
| (ALA) | | | | | |
| C18:3n6 | 0.2 (0.05 ) ^{ab} | 0.2 (0.05) ^{a} | 0.2 (0.02) ^{ab} | 0.1 (0.03) ^{b} | <0.001 |
| C20:2n6 | 0.8 (0.05) ^{a} | 0.4 (0.05) ^{b} | 0.3 (0.04) ^{c} | 0.2 (0.02) ^{c} | <0.001 |
| C20:3n6 | 2.1 (0.5) ^{a} | 1.8 (0.4) ^{a} | 1.3 (0.1) ^{b} | 1.0 (0.1) ^{ab} | <.001 |
| C20:4n6 | 9.6 (0.7) ^{a} | 9.2 (0.5) ^{a} | 17.3 (1.2) ^{b} | 8.2 (0.7) ^{a} | <0.001 |
| (AA) | | | | | |
| C20:4n3 | 0.05 (0.02) ^{a} | 0.1 (0.01) ^{b} | 0.03 (0.01) ^{c} | 0.03 (0.005) ^{ac} | <0.001 |
| C20:5n3 | 0.2 (0.02) ^{a} | 2.8 (0.2) ^{b} | 0.7 (0.2) ^{c} | 0.4 (0.1) ^{a} | 0.031 |
| (EPA) | | | | | |
| C22:4n6 | 1.4 (0.1) ^{a} | 0.8 (0.07) ^{b} | 1.5 (0.2) ^{c} | 0.9 (0.1) ^{b} | <0.001 |
| C22:5n6 | 0.2 (0.03) ^{a} | 0.2 (0.03) ^{a} | 0.5 (0.07) ^{b} | 0.1 (0.08) ^{c} | <0.001 |
| C22:5n3 | 0.7 (0.1) ^{a} | 1.9 (0.2) ^{b} | 0.5 (0.06) ^{c} | 1.3 (0.2) ^{a} | <0.001 |
| C22:6n3 | 1.9 (0.2) ^{a} | 4.0 (0.6) ^{b} | 4.8 (0.4) ^{c} | 1.3 (0.2) ^{d} | <0.001 |
| (DHA) | | | | | |
| C24:1n9 | 0.5 (0.08) ^{a} | 0.6 (0.1) ^{ab} | 0.7 (0.1) ^{b} | 0.7 (0.1) ^{b} | 0.020 |

### Example 5: Liver Function

Blood liver tests were measured at IDEXX Laboratory, Canada. Liver function tests typically include alanine transaminase (ALT) and aspartate transaminase (AST), gamma-glutamyl transferase (GGT), serum bilirubin, and albumin. For liver chemistry analysis, serum samples were collected at baseline (day 0) and at the end of trial (day 14) for measurement of standard liver chemistry including bile acids, total bilirubin, alkaline phosphatase (ALP), γ-glutamyl-transferase (GGT), and alanine aminotransferase (ALT). It should be noted that the respective data are interconnected. For example, elevations in ALT and AST in out of proportion to ALP, and bilirubin denotes a hepatocellular disease. An elevation in ALP and bilirubin in disproportion to ALT and AST would characterize a cholestatic pattern. A mixed injury pattern is defined as an elevation of alkaline phosphatase and AST/ALT levels. Isolated hyperbilirubinemia is defined as an elevation of bilirubin with normal alkaline phosphatase and AST/ALT levels. Liver function tests are performed on semi-automatic or fully automated analyzers which are based on the principle of photometry. For an overview of liver function parameters and their role in determination of liver disease see Green and Flamm, 2002, AGA technical review on the evaluation of liver chemistry tests. Gastroenterology 123 (4): 1367-1384.

### Example 5.1: Bile Flow

Bile flow was measured at terminal laparotomy, using an established approach (Muto et al., 2017, Supplemental parenteral vitamin E into conventional soybean lipid emulsion does not improve parenteral nutrition-associated liver disease in full-term neonatal piglets, JPEN 41 (4): 575-582). Following anesthesia and laparotomy the common bile duct, cystic duct and gall bladder were identified, the duct ligated and gall bladder aspirated. The common bile duct was cannulated with a standard length of polyethylene tubing. Bile flow was measured using the gravimetric method (assumed density=1) in 10-minute intervals in pre-weighed cryovials over 1 hour. Basal bile flow was defined as the average of three consecutive collections within 10% of each other.

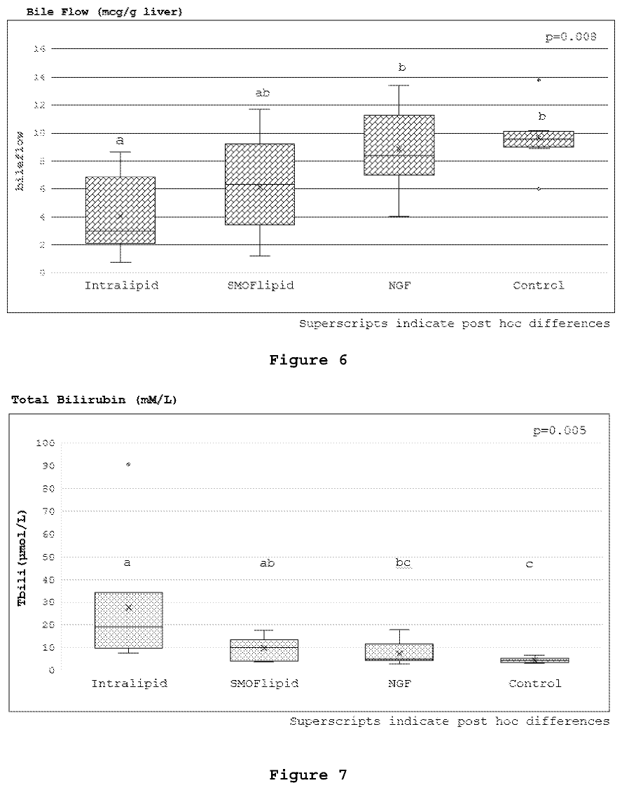

Bile flow is an important liver function and determination of bile flow is a direct marker of cholestatic liver disease. Cholestatic liver diseases are currently associated with impaired bile flow and accumulation of BAs (see Example 5.3) within the liver, resulting in hepatic inflammation and liver injury. FIG. 6 shows that bile flow is highest in the Control group, but the mean bile flow in the NGF 2 study group is clearly higher than in the comparative groups Intralipid (statistically different) and SMOFlipid (trend). This strongly underlines the positive impact of the lipid emulsion of the invention on inflammation and liver injury that otherwise result in reduced bile flow.

### Example 5.2: Bilirubin and Conjugated Bilirubin

Bilirubin is the ultimate breakdown product of hemoglobin and serves as a diagnostic marker of liver and blood disorders. Bilirubin can be toxic, especially in neonates. Unconjugated bilirubin is transported to the liver bound to albumin. Bilirubin is water-insoluble and cannot be excreted into urine. Liver injury of any cause may, for example, reduce hepatocyte cell number or influence the function of hepatocytes, such as their ability to actively transport conjugated bilirubin or bile salt into the biliary duct to allow excretion, leading to an increase in bilirubin levels. Looking at FIG. 7, the effect on the bilirubin level is very low in the NGF 2 group and basically the same as the one found in the Control group. The comparative examples Intralipid and SMOFlipid are both higher, with a more pronounced increase in bilirubin in the Intralipid group. As already indicated by bile flow and further demonstrated below, the NGF 2 group again shows a better outcome for liver injury, which is surprising given that especially ω-6 fatty acids like ARA that are contained in higher levels, for example, in certain vegetable oils like soybean oil, are currently being associated with inflammation and are generally discussed as having proinflammatory effects leading to liver damage and increased inflammation.

Unconjugated bilirubin is normally conjugated in the liver to bilirubin glucuronide and subsequently secreted into bile and the gut, respectively. An increase in conjugated bilirubin is typically associated with inflammation and liver injury (cholestasis), so any increase in conjugated bilirubin is a warning sign. The results of the study (Example 2) for conjugated bilirubin are shown in FIG. 8. Surprisingly, the NGF 2 group shows very low levels of conjugated bilirubin which is basically at the same level as the Control group. The comparative lipid emulsions result in clearly higher levels of conjugated bilirubin. Again, this is a surprising result as discussed above, and highlights an unexpectedly positive impact of the lipid emulsions according to the invention on liver injury and inflammation.

### Example 5.3: Bile Acids

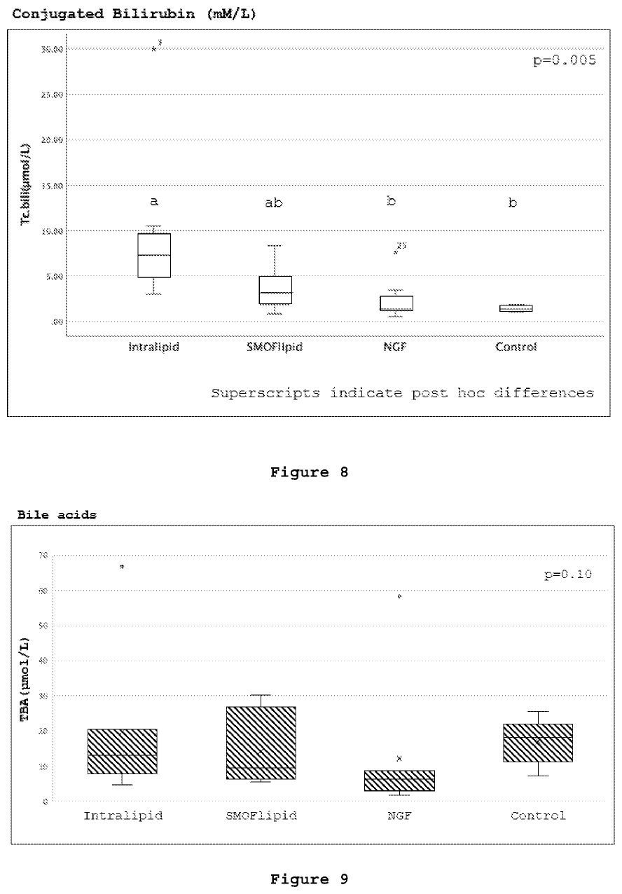

Bile acids (BAs) are the primary end metabolic product of cholesterol, having detergent-like activities and playing important roles in lipid absorption and utilization of lipids. As natural detergents, BAs might disrupt lipid components of cell membranes and advocate oxidative damage. Hence, accumulation of BAs within hepatocytes leads to apoptosis or necrosis of hepatocytes, which is principally responsible for cholestatic liver damage (Li and Lu, 2018, Therapeutic Roles of Bile Acid Signaling in Chronic Liver Diseases, Journal of Clinical and Translational Hepatology 6:425-430). Cholestatic liver diseases are currently associated with impaired bile flow (see Example 5.1) and accumulation of BAs within the liver, resulting in hepatic inflammation and liver injury. As can be seen in FIG. 9, bile acid levels are very low in study group NGF 2 according to the invention and actually lowest compared to all tested groups, underlining the surprisingly positive impact of the lipid emulsion as disclosed herein on inflammation and liver injury.

### Example 5.4: Aminotransferase (ALT) and Aspartate Transaminase (AST) Development

ALT and AST are cytosolic enzymes that are found in high concentrations in the liver. Hepatocellular injury and not necessarily cell death triggers the release of these enzymes into circulation. For assessing the impact of the lipid emulsion of the invention on the liver status of the animals tested as described in Example 2, aminotransferase (ALT) activity in the plasma of tested animals was tested. As mentioned above, liver damage may be established by increased aminotransferase levels. ALT activity can be determined with an ADVIA 1800 blood biochemistry analyzer/IFCC modified (Siemens) according to standard protocols.

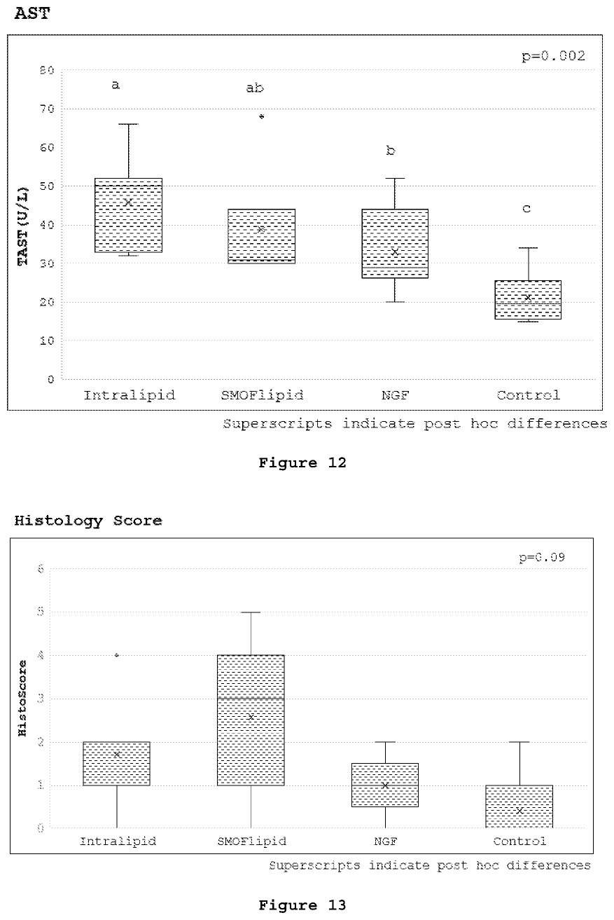

To summarize the results of the experiments, FIG. 11 shows the development of ALT activity in the tested groups. In comparison, ALT activity does not increase in all study groups over the study period and is found to be highest in the Control group. FIG. 12 shows that AST levels are higher for all study groups compared to the Control, but are lowest for NGF 2, indicating its positive impact on the liver.

### Example 5.5: Gamma-Glutamyl Transferase (GGT)

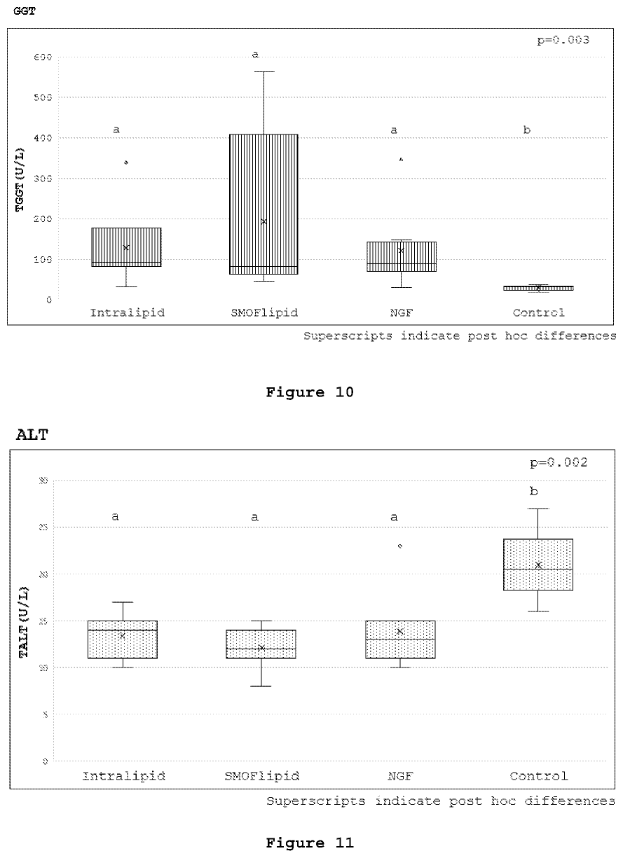

Glycoprotein gamma-glutamyltransferase (GGT) is located on membranes of cells with high secretory or absorptive activities. Its primary function is to catalyze the transfer of a gamma-glutamyl group from peptides to other amino acids. GGT activity level in human children may be a reliable index of bile duct damage. As can be seen in FIG. 10, GGT activity is lowest in the Control group as could be expected and is about the same in the study groups, with stably low values for NGF-2 in comparison to the SMOFlipid group.

TABLE XI
Overview Liver Outcomes (mean/SD; median/IQR)

| | | | | | P value |
|---|---|---|---|---|---|
| | Intralipid | SMOFlipid | NGF 2 | Control | Kruskal |
| | (n = 7) | (n = 7) | (n = 8) | (n = 8) | Wallis |
| Bile flow | 4.0 (2.8) | 6.1 (3.7) | 8.8 (2.9) | 9.7 (2.1) | 0.045 |
| (μg/kg/min) # | 3.0 (4.8) ^{a} | 6.3 (5.8) ^{a} | 8.4 (4. 3) ^{ab} | 9.6 (1.2) ^{b} | |
| Total Bilirubin | 27.7 (29.2) | 9.8 (5.0) | 7.4 (5.3) | 4.5 (1.2) | 0.006 |
| (μmol/L) | 19.3 (24.5) ^{a} | 10.0 (9.6) ^{ab} | 5.0 (7.1) ^{bc} | 4.4 (1.9) ^{c} | |
| Conj Bilirubin | 9.9 (9.2) | 3.7 (2.7) | 2.3 (2.3) | 1.4 (0.3) | 0.006 |
| (μmol/L) | 7.4 ( 6.6) ^{a} | 3.1 (5.0) ^{ab} | 1.4 (2.0) ^{b} | 1.4 (0.6) ^{b} | |
| Bile Acids | 19.7 (21.3) | 14.2 (10.2) | 12.2 (18.8) | 17.2 (6.3) | 0.092 |
| (μmol/L) | 13.1 (12.7) | 9.5 (20.4) | 6.4 (5.7) | 18.2 (10.9) | |
| Alk Phos | 1007 (352) | 659 (126) | 684 (377) | 660 (238) | 0.16 |
| (U/L) | 908 (498) | 641 (77) | 538 (254) | 618 (446) | |
| GGT (U/L) | 130 (102) | 193 (207) | 123 (98) | 31 (7) | 0.007 |
| | 93 (96) ^{a} | 84 (345) ^{a} | 91 (74) ^{a} | 34 (9) ^{b} | |
| AST (U/L) | 46 (12) | 39 (14) | 33 (11) | 18 (5) | 0.004 |
| | 50 (19) ^{a} | 31 (14) ^{a} | 29 (18) ^{a} | 17 (7) ^{b} | |
| ALT (U/L) | 13 (2) | 12 (2) | 14 (4) | 21 (4) | 0.001 |
| | 14 (4) ^{a} | 12 (3) ^{a} | 13 (4) ^{a} | 20 (6) ^{b} | |

### Example 6: Histology

Liver histology was assessed from tissue sections fixed in 10% neutral buffered formaldehyde, embedded in paraffin and stained using standard techniques with hematoxylin and eosin. Blinded to group, a veterinary pathologist performed the histopathological assessment of cholestasis, steatosis, inflammatory infiltration and fibrosis, in at least 10 “classic” lobules that have a clear central vein. A scoring system was applied that is based on a published human hepatitis C scoring system for liver cirrhosis (Lim et al, 2016, Glucagon-like Peptide-2 improves cholestasis in parenteral nutrition associated liver disease in neonates. JPEN 40 (1): 14-21. ORO staining of formalin embedded liver tissue was performed at Prairie Diagnostics using a modified scoring system as previously published (Isaac et al., 2019, Mixed Lipid, Fish Oil, and Soybean Oil Parenteral Lipids Impact Cholestasis, Hepatic Phytosterol, and Lipid Composition. Journal of Pediatric Gastroenterology and Nutrition 68 (6): 861-867). FIG. 13 shows that parenteral nutrition based on the NGF 2 lipid emulsion scores lowest of all parenterally fed groups and is closest to the Control group with normally raised piglets, indicating a positive impact on avoiding or reducing the development of liver damage and improving the healthy development of the liver tissue, which is of special importance for pediatric patients, and especially for pre-term and extremely pre-term babies.

1. A lipid emulsion for parenteral nutrition comprising an aqueous phase and about 5% to about 35% by weight of an oil phase based on the total weight of the lipid emulsion (w/w), wherein the lipid emulsion comprises
from about 2.0 g/L to about 15.0 g/L of DHA;
from about 0 g/L to about 1.2 g/L of EPA; and
from about 5.0 g/L to about 20.0 g/L of ARA.

2. The lipid emulsion according to claim 1, wherein the ratio of DHA:ARA is from 1:0.5 to 1:2.

3. The lipid emulsion according to claim 1, further comprising choline in an amount of from 0.5 g/L to 5.0 g/L.

4. The lipid emulsion according to claim 3, wherein the choline is selected from choline chloride and glycerophosphocholine.

5. The lipid emulsion according to claim 1, wherein the lipid emulsion contains phytosterols in an amount not exceeding 200 mg/L of the lipid emulsion.

6. The lipid emulsion according to claim 1, wherein the lipid emulsion contains phytosterols in an amount not to exceed 140 mg/mL.

7. The lipid emulsion according to claim 1, wherein the ratio of DHA to EPA is from 30:1 to 10:1.

8. The lipid emulsion according to claim 1, comprising linoleic acid in a concentration of from 25 to 50 g/L.

9. The lipid emulsion according to claim 1, comprising α-tocopherol in a concentration of from 100-300 mg/L.

10. The lipid emulsion according to claim 1, wherein the ratio of ω-6:0-3 fatty acids is from 4:1 to 2:1.

11. The lipid emulsion according to claim 1 for the treatment of pediatric patients.

12. A multi-chamber bag for providing a parenteral nutrition formulation, wherein the multi-chamber bag comprises a chamber containing a lipid emulsion according to claim 1.

13. A method of treating patients who require parenteral nutrition when oral and enteral nutrition is not possible, insufficient, or contraindicated, with a lipid emulsion according to claim 1.

14. A method of preventing or ameliorating liver damage and/or inflammation in pediatric patients requiring parenteral nutrition, wherein the pediatric patients are parenterally administered a composition comprising a lipid emulsion according to claim 1.

15. A method of improving the fatty acid profile of pediatric patients requiring parenteral nutrition in the patients' plasma and/or tissues, wherein the pediatric patients are parenterally administered a composition comprising a lipid emulsion according to claim 1.

16. The method of claim 15, wherein the tissues comprise the tissue of lung, liver and/or retina.
